(19) United States
(12) Patent Application Publication
(10) Pub. No.: US 2014/0336397 A1
(43) Pub. Date: Nov. 13, 2014

(54) METHODS FOR THE SYNTHESIS OF 13C LABELED DHA AND USE AS A REFERENCE STANDARD

(71) Applicant: PHENOMENOME DISCOVERIES INC., Saskatoon, Saskatchewan (CA)

(72) Inventors: M. Amin Khan, Morgan Hill, CA (US); Paul L. Wood, Harrogate, TN (US); Dayan Goodenowe, Saskatoon (CA)

(21) Appl. No.: 14/359,097

(22) PCT Filed: Nov. 16, 2012
(86) PCT No.: PCT/CA2012/001053

§ 371 Date: May 17, 2014

Related U.S. Application Data
(60) Provisional application No. 61/561,225, filed on Nov. 17, 2011.

(51) Int. Cl. C07C 51/09; A61K 49/00; C07C 57/03; C07B 59/00

(52) U.S. Cl. CPC C07C 51/09; C07B 59/001; A61K 49/00; C07C 57/03
USPC 554/145; 554/224

(57) ABSTRACT

A method for preparing ¹³C labeled docosahexaenoic acid (DHA) represented by Formula A: The method comprises the conversion of 2-pentyn-1-ol to ¹³C labeled DHA by reaction with propargyl alcohol, ¹³C labeled propargyl alcohol and methyl pent-4-ynoate. The various steps involved include tosylation, coupling, bromination, selective hydrogenation and ester hydrolysis to obtain the final product.

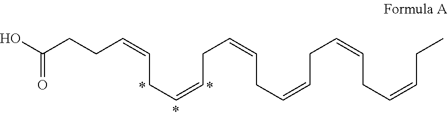 [SMILES: CC/C=C\C/C=C\C/C=C\C/C=C\C/C=C\C/C=C\CCC(=O)O]

### FIELD OF INVENTION

The present invention relates to methods for the chemical synthesis of fatty acids, and specifically, to methods for the chemical synthesis of ¹³C labeled fatty acids such as docosahexaenoic acid.

### BACKGROUND OF THE INVENTION

Docosahexaenoic acid (DHA) is an omega-3 unsaturated fatty acid, containing a chain-terminating carboxylic acid group and six cis-double bonds in a 22-carbon straight chain. Its trivial name is cervonic acid, its systematic name is all-cis-docosa-4,7,10,13,16,19-hexa-enoic acid, and its shorthand name is 22:6w3 in the nomenclature of fatty acids. Its chemical structure can be represented as follows:

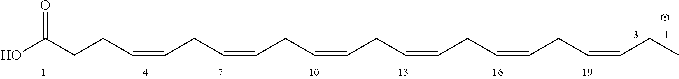 [SMILES: CC/C=C\C/C=C\C/C=C\C/C=C\C/C=C\C/C=C\CCC(=O)O]

DHA is essential for the growth, functional development and healthy maintenance of brain function and is required throughout life from infancy through aging (Horrocks, L. A. and Y. K. Yeo. Pharmacol. Res. 40(3):211-225 (1999)). It is derived from the essential precursor linolenic acid (LNA, 18:3w3). DHA is the main end-product of LNA after successive desaturations and elongations, a metabolic cascade that is assumed to be weak in humans (Burdge G C, Jones A E, Wootton S A (2002) Eicosapentaenoic and docosapentaenoic acids are the principal products of alphalinolenic acid metabolism in young men. Br J Nutr 88:355-363; Brenna J T, Salem N Jr, Sinclair A J, Cunnane S C (2009) Alphalinolenic acid supplementation and conversion to n-3 long-chain polyunsaturated fatty acids in humans. Prostaglandins Leukot Essent Fatty Acids 80:85-91).

DHA has been attributed to physiological effects such as blood lipid reduction, anticoagulant effect, carcinostatic effect, and improvement in visual functions. DHA was found to inhibit growth of human colon carcinoma cells (Kato T, Hancock R L, Mohammadpour H, McGregor B, Manalo P, Khaiboullina S, Hall M R, Pardini L, Pardini R S (2002). “Influence of omega-3 fatty acids on the growth of human colon carcinoma in nude mice”. Cancer Lett. 187 (1-2): 169-77). Dietary DHA may reduce the risk of heart disease by reducing the level of blood triglycerides in humans. Further, DHA deficiencies are associated with fetal alcohol syndrome, attention deficit hyperactivity disorder, cystic fibrosis, phenylketonuria, unipolar depression, aggressive hostility and adrenoleukodystrophy. In contrast, increased intake of DHA has been shown to be beneficial or have a positive effect in inflammatory disorders (e.g., rheumatoid arthritis), Type II diabetes, hypertension, atherosclerosis, depression, myocardial infarction, thrombosis, some cancers and for prevention of the onset of degenerative disorders such as Alzheimer's disease (U.S. Pat. No. 7,550,286 B2).

Due to its various physiological effects, DHA is also administered as a dietary supplement.

However, the mechanism of action as well as the fate of DHA in the body is still not completely understood. Therefore, it is of interest to study the metabolism of DHA in the body. Also, if DHA is to be administered as a dietary supplement, the fate of the DHA supplement administered needs to be known.

Thus developing stable metabolic tracers for DHA is needed. To this end, ¹³C labeled DHA has been utilized as a metabolic tracer to study the uptake and metabolism of DHA. Further, ¹³C labeled DHA was also used to study the placental transfer of DHA from mother to fetus (In vivo investigation of the placental transfer of (13)C-labeled fatty acids in humans. Larquè E, Demmelmair H, Berger B, Hasbargen U and Koletzko B.; J Lipid Res. 44(1):49-55 (2003)).

Currently known methods of producing ¹³C labeled DHA include biosynthetic production. In such methods, micro-organisms capable of producing DHA are cultured on ¹³C labeled precursors for DHA such as ¹³C glucose, ¹³C malonyl CoA (Biosynthetic production of universally (13)C-labeled polyunsaturated fatty acids as reference materials for natural health product research. Le P M, Fraser C, Gardner G, Liang W W, Kralovec J A, Cunnane S C, Windust A J, Anal Bioanal Chem. 389(1):241-9 (2007)). The DHA synthesized is then extracted from such cultures. Another way of studying metabolism of ¹³C labeled DHA is by synthesis of phospholipids such as phosphatidyl choline in which ¹³C labeled DHA is present at the sn-2 position. This ¹³C DHA is released from the phospholipid by phospholipase A2 present in the body. Then the fate of DHA can be followed (Blood compartmental metabolism of docosahexaenoic acid (DHA) in humans after ingestion of a single dose of [(13)C]DHA in phosphatidylcholine. Lemaitre-Delaunay D, Pachiaudi C, Laville M, Pousin J, Armstrong M, Lagarde M., J Lipid Res., 40(10):1867-74 (1999)). ¹³C labeled DHA can also similarly be incorporated into triglycerides (Human plasma albumin transports [13C]docosahexaenoic acid in two lipid forms to blood cells. Brossard N, Croset M, Normand S, Pousin J, Lecerf J, Laville M, Tayot J L, Lagarde M. J Lipid Res. 38(8):1571-82. (1997)). However, synthesis of such phospholipids also depends on micro-organisms capable of synthesizing the phospholipid. Thus, the methods known so far are expensive and cumbersome as they involve complex extraction steps. Also, desired product is obtained in low yields.

### SUMMARY OF THE INVENTION

There is accordingly a need for new and improved methods for synthesizing ¹³C labeled fatty acids, such as but not limited to DHA. The present invention aims to provide such a method.

In an aspect of the invention, a process is provided for preparing a ¹³C labeled fatty acid represented by Formula (i):

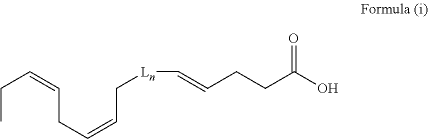

wherein L is —[CH═CH—CH₂]—, and n is 0 to 6, preferably 1 to 4, more preferably 3, and the compound comprises at least one ¹³C labeled carbon residue. The process comprises:

(a) converting 2-pentyn-1-ol into a tosylate of Formula (ii), e.g by reaction with tosyl chloride (TsCl):

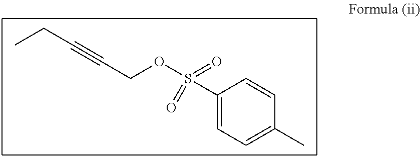 [SMILES: CCC#CCOS(=O)(=O)c1ccc(C)cc1]

(b) reacting the compound of Formula (ii) with propargyl alcohol in a coupling reaction, and optionally carrying out one or more additional steps of brominating followed by coupling with propargyl alcohol, to obtain a compound represented by Formula (iii):

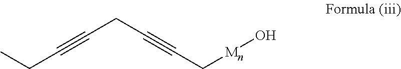

wherein M is —[C≡C—CH₂]—, and n is as defined above,
(c) carrying out a selective reduction of the compound represented by Formula (iii) to obtain a compound represented by Formula (iv):

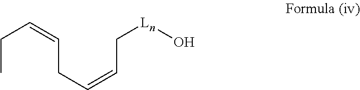

wherein L and n are as defined above,
(d) brominating the compound of Formula (iv) to produce a compound represented by Formula (v):

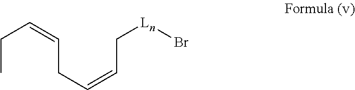

wherein L and n are as defined above,
(e) coupling the compound represented by Formula (v) with methyl pent-4-ynoate to obtain a compound represented by Formula (vi):

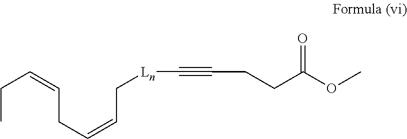

(f) carrying out a selective reduction of the compound represented by Formula (vi) to obtain a compound represented by Formula (vii):

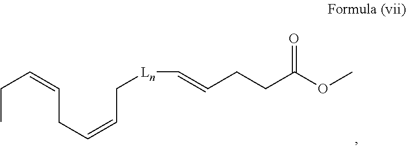

and
(g) ester-hydrolyzing the compound represented by Formula (vii) to obtain the compound represented by Formula (i),
wherein the propargyl alcohol used in at least one of the coupling reactions carried out in (b) is labeled with ¹³C at C₁, C₂, or C₃ of the propargyl alcohol, or a combination thereof.

In one embodiment of the invention, a process is provided for preparing a ¹³C labeled DHA represented by Formula A:

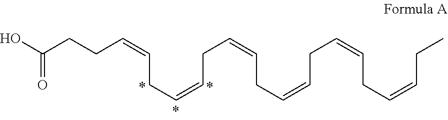 [SMILES: CC/C=C\C/C=C\C/C=C\C/C=C\C/C=C\C/C=C\CCC(=O)O]

where * represents a ¹³C labeled carbon residue.

In this process, 2-pentyn-1-ol of Formula 1:

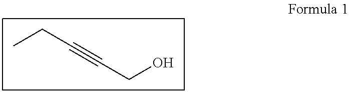

is reacted with tosyl chloride (TsCl) to obtain a compound represented by Formula 2:

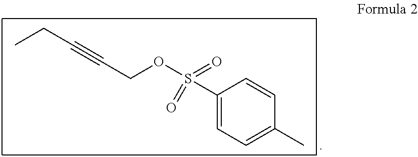 [SMILES: CCC#CCOS(=O)(=O)c1ccc(C)cc1]

In certain non-limiting embodiments, the compound of Formula 2 can be obtained with a yield of 60-68%.

The compound of Formula 2 is then coupled with propargyl alcohol to produce a compound represented by Formula 3:

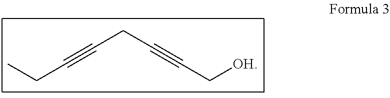

In certain non-limiting embodiments, the compound of Formula 3 can be obtained with a yield of 93-99%.

The compound of Formula 3 is then reacted with PBr₃ to produce a compound represented by Formula 4:

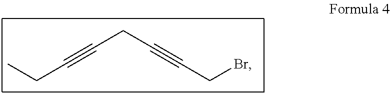

and the resulting compound is coupled with propargyl alcohol to obtain a compound represented by Formula 5:

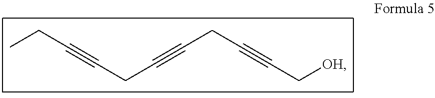

In certain non-limiting embodiments, the compound of Formula 5 is obtained with a yield of 52-62%.

The compound represented by Formula 5 is reacted with PBr₃ to a produce a compound represented by Formula 6:

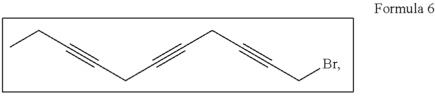

and the resulting compound is coupled with propargyl alcohol to obtain a compound represented by Formula 7:

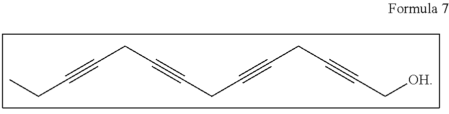

In certain non-limiting embodiments, the compound of Formula 7 is obtained with a yield of 27-37%.

The resulting compound of Formula 7 is reacted with PBr₃ to produce a compound represented by Formula 8:

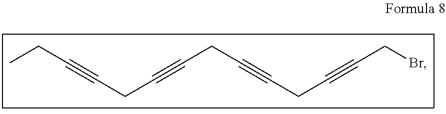

and the resulting compound is coupled with ¹³C labeled propargyl alcohol to obtain a compound represented by Formula 9:

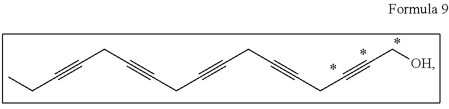 [SMILES: CCC#CCC#CCC#CCC#CCC#CCO]

where * represents a ¹³C labeled carbon residue.

In certain non-limiting embodiments, the compound of Formula 9 is obtained with a yield of 45-55%.

Selective reduction of the compound represented by Formula 9 is then carried out to obtain a compound represented by Formula 10:

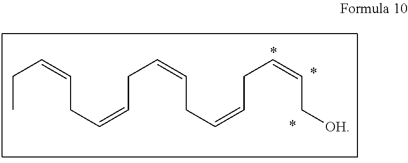 [SMILES: CC/C=C\C/C=C\C/C=C\C/C=C\C/C=C\CO]

In certain non-limiting embodiments, the compound of Formula 10 is obtained with a yield of 63-73%.

The compound of Formula 10 is then reacted with PBr₃ to produce a compound represented by Formula 11:

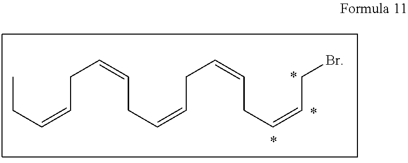 [SMILES: CC/C=C\C/C=C\C/C=C\C/C=C\C/C=C\CBr]

The compound represented by Formula 11 is then reacted with methyl pent-4-ynoate in a coupling reaction to produce a compound represented by Formula 12:

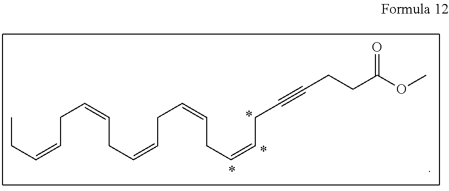 [SMILES: CC/C=C\C/C=C\C/C=C\C/C=C\C/C=C\CC#CCCC(=O)OC]

In certain non-limiting embodiments, the compound of Formula 12 is obtained with a yield of 49-59%.

Selective reduction of the compound represented by Formula 12 is then carried out to produce a compound represented by Formula 13:

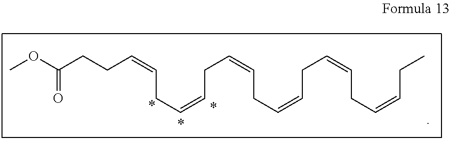 [SMILES: CC/C=C\C/C=C\C/C=C\C/C=C\C/C=C\C/C=C\CCC(=O)OC]

In certain non-limiting embodiments, the compound of Formula 13 is obtained with a yield of 75-85%.

Finally, the compound represented by Formula 13 is ester-hydrolyzed to produce the compound of Formula A. In certain non-limiting embodiments, the compound of Formula 1 is obtained with a yield of 82-92%.

In a preferred, yet non-limiting embodiments of the synthetic process, one or more of the bromination reactions for producing compounds of Formulas 4, 6, 8 and 11 are carried out in presence of pyridine and dichloromethane. The temperature of the bromination reaction is also preferred to be from about 0° C. to about room temperature.

In yet another preferred embodiment, which is non-limiting, one or more of the coupling reactions for production of the compounds represented by Formulas 5, 7, 9 and 12 are carried out in the presence of CuI, tetrabutylammonium iodide (TBAI) in dry N,N-dimethylformamide (DMF). The temperature of the coupling reaction is also preferred to be from about 0° C. to about room temperature.

In further non-limiting embodiments, one or more of the hydrogenation reactions for production of the compounds represented by Formulas 10 and 13 are carried out at about room temperature, in an H₂ atmosphere, and using a catalyst such as but not limited to Lindlar's catalyst.

In another non-limiting embodiment, LiOH is used for ester hydrolysis of the compound represented by Formula 13, in the presence of THF/H₂O (3:1), to obtain the compound represented by Formula 1.

### BRIEF DESCRIPTION OF THE DRAWINGS

These and other features of the invention will become more apparent from the following description in which reference is made to the appended drawings wherein:

FIG. 1 illustrates the NMR spectra of the ¹³C labeled DHA of Formula 1, prepared by an embodiment of a synthetic process of the present invention;

FIG. 2 illustrates the LC chromatogram of the ¹³C labeled DHA of Formula 1, prepared by an embodiment of a synthetic process of the present invention; and

FIG. 3 illustrates the LC-MS results of the ¹³C labeled DHA of Formula (A shows the LC trace, and B shows the MS results), prepared by an embodiment of a synthetic process of the present invention.

### DETAILED DESCRIPTION

The present invention provides a useful synthetic process for preparing ¹³C labeled fatty acids. The process involves preparing a ¹³C labeled fatty acid represented by Formula (i):

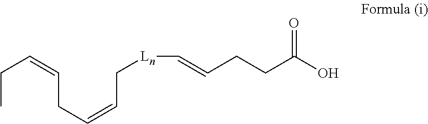

wherein L is —[CH═CH—CH₂]—, and n is 0 to 6, preferably 1 to 4, more preferably 3, and the fatty acid comprises at least one ¹³C labeled carbon residue. The process comprises:
(a) converting 2-pentyn-1-ol into a tosylate of Formula (ii), e.g by reaction with tosyl chloride (TsCl):

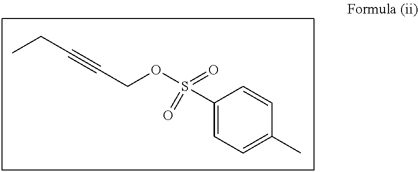 [SMILES: CCC#CCOS(=O)(=O)c1ccc(C)cc1]

(b) reacting the compound of Formula (ii) with propargyl alcohol in a coupling reaction, and optionally carrying out one or more additional steps of brominating followed by coupling with propargyl alcohol, to obtain a compound represented by Formula (iii):

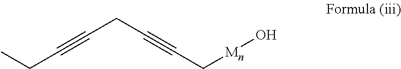

wherein M is —[C≡C—CH₂]—, and n is as defined above,
(c) carrying out a selective reduction of the compound represented by Formula (iii) to obtain a compound represented by Formula (iv):

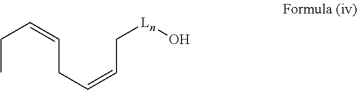

wherein L and n are as defined above,
(d) brominating the compound of Formula (iv) to produce a compound represented by Formula (v):

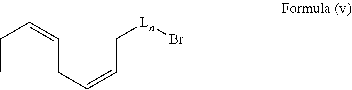

wherein L and n are as defined above,
(e) coupling the compound represented by Formula (v) with methyl pent-4-ynoate to obtain a compound represented by Formula (vi):

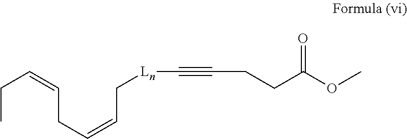

(f) carrying out a selective reduction of the compound represented by Formula (vi) to obtain a compound represented by Formula (vii):

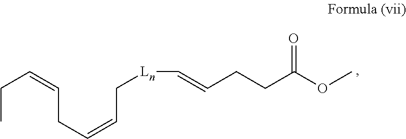

and
(g) ester-hydrolyzing the compound represented by Formula (vii) to obtain the compound represented by Formula (i),
wherein the propargyl alcohol used in at least one of the coupling reactions carried out in (b) is labeled with ¹³C at C₁, C₂, or C₃ of the propargyl alcohol, or a combination thereof.

In one non-limiting embodiment of the invention, a process is provided for preparing DHA, for example as represented below by Formula A:

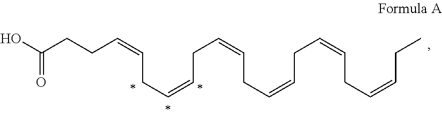 [SMILES: CC/C=C\C/C=C\C/C=C\C/C=C\C/C=C\C/C=C\CCC(=O)O]

where * represents a ¹³C labeled carbon residue.

This synthetic route can, in certain preferred embodiments, yield high purity of ¹³C fatty acids, such as DHA, and at reduced cost as compared to other methods through the use of generally abundant and inexpensive reagents. The process also has the advantage that, in certain embodiments, no downstream processing is required.

It will be appreciated by those skilled in the art that each of the embodiments of the invention described herein may be utilized individually or combined in one or more manners different than the ones disclosed above for the production of ¹³C labeled fatty acids, including DHA. In addition, those skilled in the art will be able to select a suitable temperature in view of the reaction conditions being used, in further embodiments of the invention encompassed herein.

The literature referred to herein establishes knowledge that is available to those with skill in the art. Unless otherwise defined, all technical and scientific terms used herein have the same meaning as commonly understood by one of ordinary skill in the art to which this invention relates. All references cited herein are hereby incorporated by reference to the same extent as if each was specifically and individually incorporated by reference.

Although methods and materials similar or equivalent to those described herein can be used in the practice or testing of the present invention, the preferred methods and materials are described herein. In the case of inconsistencies, the present disclosure, including definitions, will control. In addition, the materials, methods, and examples are illustrative only and are not intended to be limiting.

The term “about” is used herein to mean approximately, in the region of, roughly, or around. When the term “about” is used in conjunction with a numerical range, it modifies that range by extending the boundaries above and below the numerical values set forth. The term “comprises” is used herein to mean “includes, but is not limited to.”

The following abbreviations are used throughout the specification:

### CuI: Copper Iodide

### DHA: Docosahexanoic Acid

### DCM: Dichloromethane

### DMF: Dimethylformamide

### EtOAc: Ethyl Acetate

### HCl: Hydrochloric Acid

### K₂CO₃: Potassium Carbonate

### KOH: Potassium Hydroxide

### MeOH: Methanol

### NaHCO₃: Sodium Carbonate

### Na₂SO₄: Sodium Sulphate

### PBr3: Phosphorus Tribromide

### Py: Pyrimidine

### TBAI: Tetrabutylammonium Iodide

### THF: Tetrahydrofuran

### TsCl: Tosyl Chloride

In one embodiment of the invention, a 13-step chemical synthetic process for preparing ¹³C DHA of Formula A is provided. The synthetic process is depicted below in Scheme A.

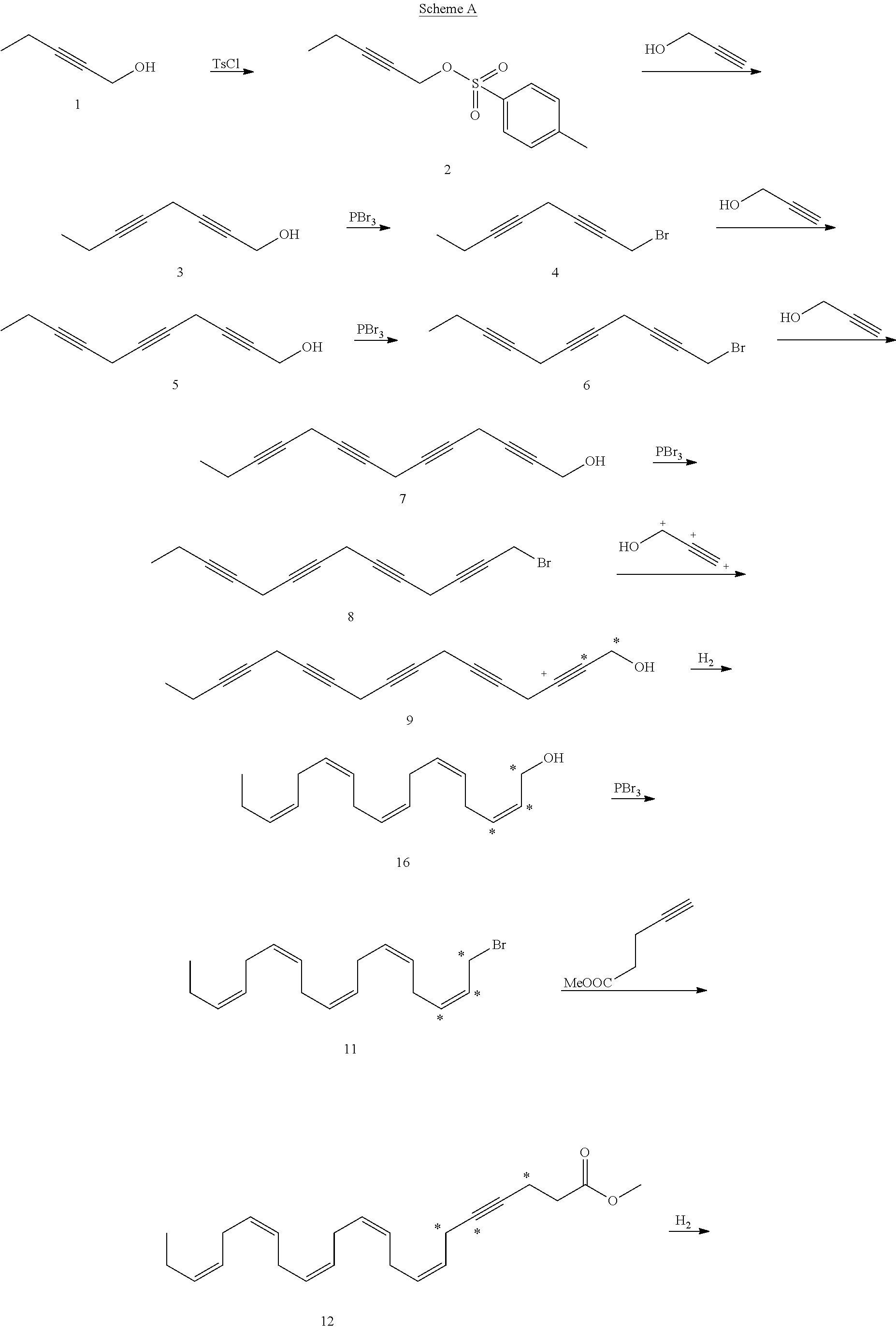

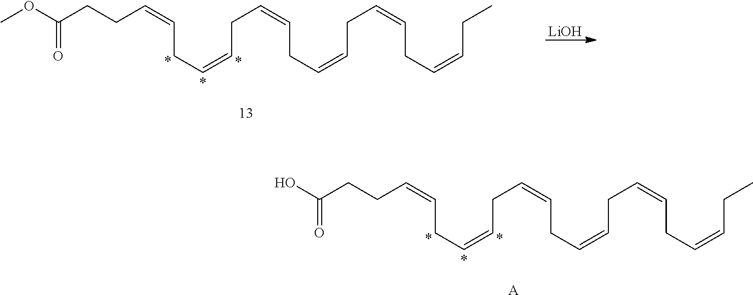

In this synthetic process, 2-Pentyn-1-ol of Formula 1 is used as a starting material, wherein the alcohol group in 2-Pentyn-1-ol is converted to tosyl as represented by Formula 2, using TSCl/KOH. The resulting compound of Formula 2 is coupled with propargyl alcohol using CuI/K₂CO₃/TBAI to obtain a compound represented by Formula 3 in good yield. The compound of Formula 3 is coupled with propargyl alcohol, via a bromide represented by Formula 4 to obtain a compound represented by Formula 5. The compound of Formula 5 is coupled with propargyl alcohol, via a bromide represented by Formula 6 to obtain a compound represented by Formula 7. The compound represented by Formula 7 is further coupled with a ¹³C labeled propargyl alcohol via the bromide represented by Formula 8 to obtain a compound represented by Formula 9. The resulting compound of Formula 9 is selectively reduced, e.g. using Lindlar's catalyst, to produce a compound represented by Formula 10 which is then coupled with methyl pent-4-ynoate via a bromide represented by Formula 11 to obtain a compound represented by Formula 12. The compound represented by Formula 12 is selectively reduced, e.g. using a Lindlar's catalyst, to produce a compound represented by Formula 13, which is ester hydrolyzed, e.g. using LiOH, to produce the ¹³C-labeled DHA of Formula A.

In yet another embodiment of the invention, an alternate, 12-step chemical synthetic process for preparing ¹³C DHA of Formula A is provided. The synthetic process is depicted below in Scheme B.

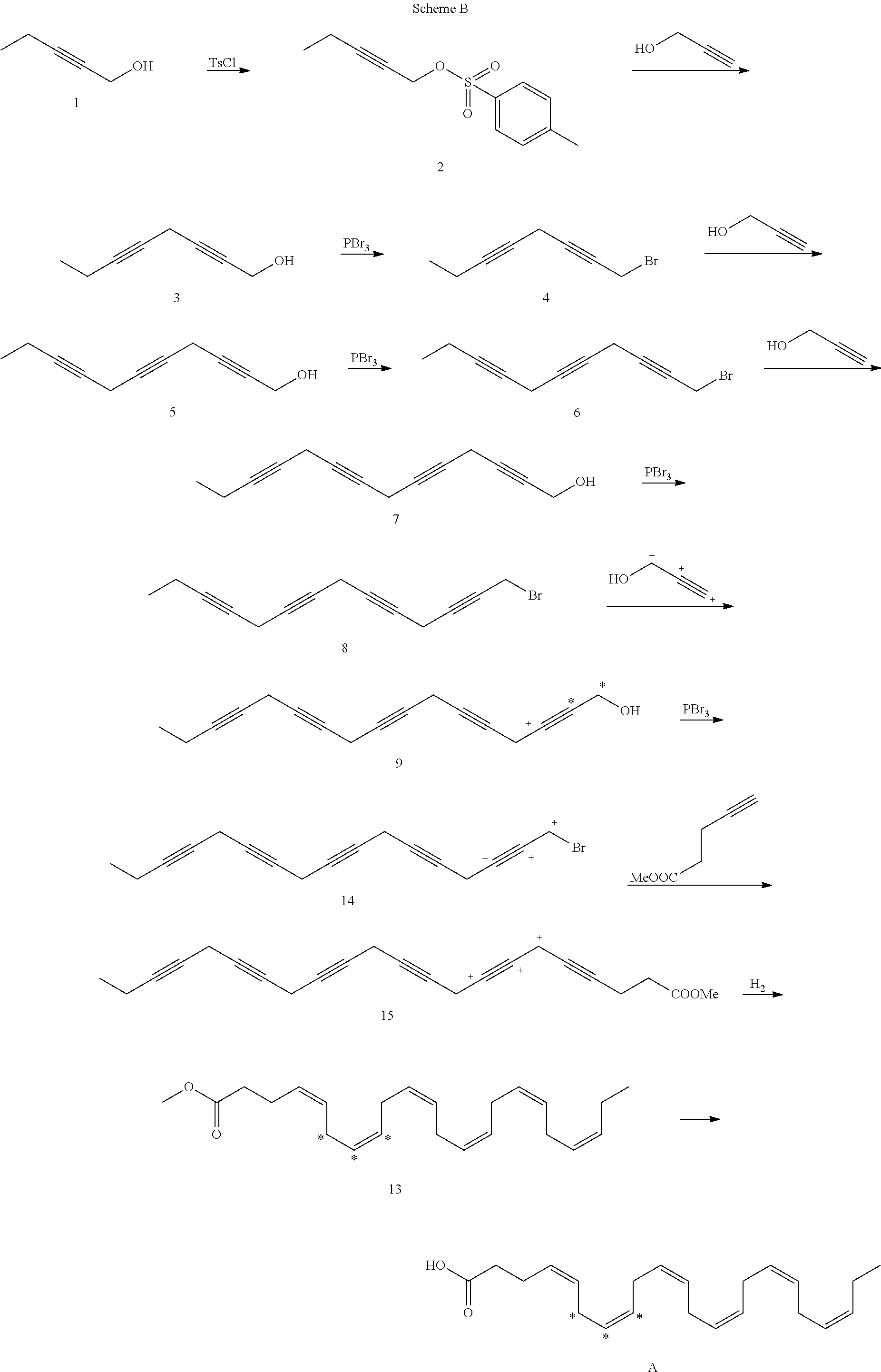

In the alternate synthetic process, 2-Pentyn-1-ol of Formula 1 is used as a starting material, wherein the alcohol group in 2-Pentyn-1-ol is converted to tosyl as represented by Formula 2, using TSCl/KOH. The resulting compound of Formula 2 is coupled with propargyl alcohol using CuI/K₂CO₃/TBAI to obtain a compound represented by Formula 3 in good yield. The compound of Formula 3 obtained is coupled with propargyl alcohol, via a bromide represented by Formula 4 to obtain a compound represented by Formula 5. The compound represented by Formula 5 is coupled with propargyl alcohol, via a bromide represented by Formula 6 to obtain a compound represented by Formula 7. The compound represented by Formula 7 is further coupled with a ¹³C labeled propargyl alcohol via the bromide represented by Formula 8 to obtain a compound represented by Formula 9. The resulting compound of Formula 9 is then coupled with methyl pent-4-ynoate via a bromide represented by Formula 14 to obtain a compound represented by Formula 15. The compound represented by Formula 15 is selectively reduced using a Lindlar's catalyst to produce a compound represented by Formula 13, which is ester hydrolyzed, e.g. using LiOH, to produce the ¹³C-labeled DHA of Formula A.

### EXAMPLES

The following provides examples of certain preferred embodiments of the synthetic process described herein for producing the ¹³C labeled DHA of Formula A. The process is depicted below in Scheme C.

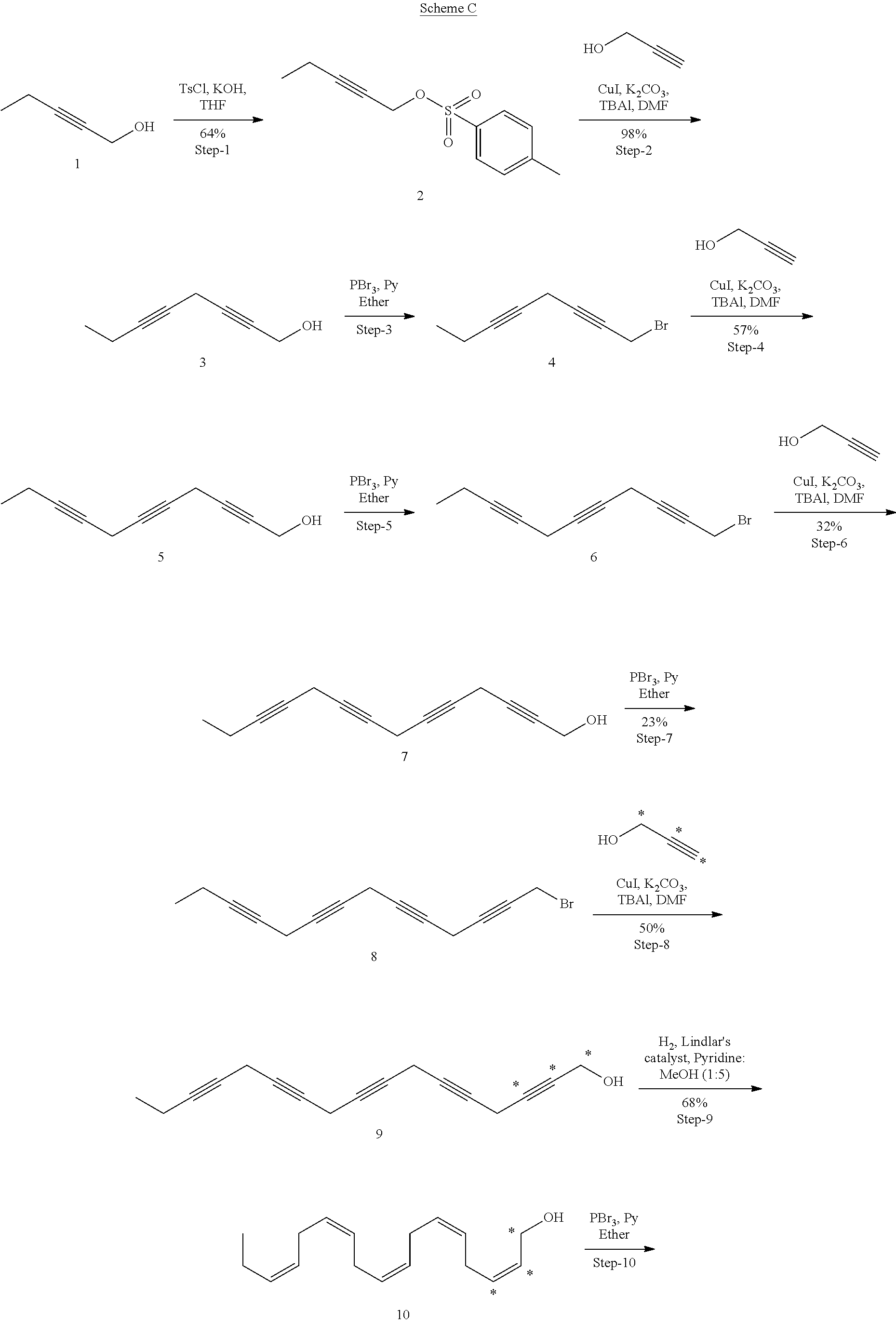

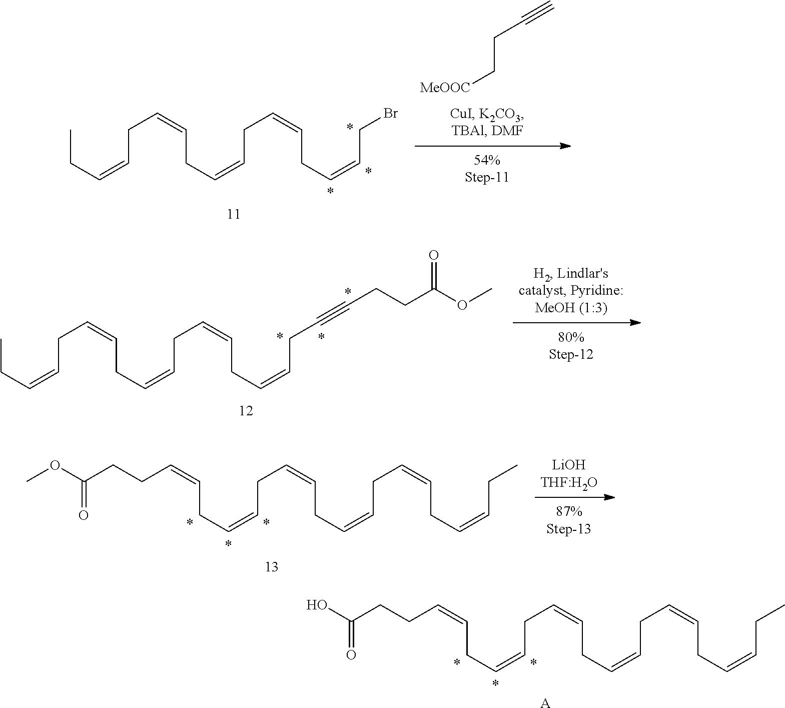

### Example 1

### Synthesis of ¹³C DHA Using 13-Step Chemical Synthetic Process

In the 13-step chemical synthetic process for preparing ¹³C DHA of Formula A, 2-Pentyn-1-ol of Formula 1 and tosyl chloride are used as the starting materials. Each of the steps in the chemical synthetic process are described in detail below.

### Preparation of Compound of Formula 2 (Pent-2-ynyl 4-methylbenzenesulfonate)

In the first step of the synthetic process, 2-pentyn-1-ol of Formula 1 is converted to the tosyl compound represented by Formula 2 using tosyl chloride in the presence of KOH. The yield of the compound ranges from 60-68%. The reaction scheme involved in this process is as follows:

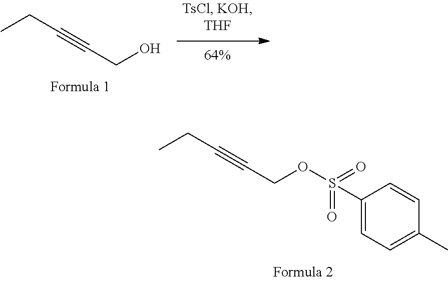

In an exemplary embodiment, the raw materials used for this step are illustrated in Table 1:

TABLE 1

| S. | Name of the | | | M. | | | |
|---|---|---|---|---|---|---|---|
| No. | Material | Qty. | | Wt. | Moles | Mole Ratio | |
| 1. | 2-Pentyn- | 60 | g | 84.12 | 0.71 | 1 | |
| | 1-ol | | | | | | |
| 2. | Tosyl | 142.9 | g | 190.65 | 0.75 | 1.06 | |
| | Chloride | | | | | | |
| | (TsCl) | | | | | | |
| 3. | KOH | 79.9 | g | 56.11 | 1.42 | 2 | |
| 4. | THF | 420 | mL | 72.11 | — | 7 | vol. |
| 5. | Ethyl | 600 | mL | 88.11 | — | 10 | vol. |
| | Acetate | | | | | | |
| 6. | Water | 2 × 100 | mL | 18 | — | 2 × 1.67 | vol. |
| 7. | Brine | 2 × 50 | mL | — | — | 2 × 0.83 | vol. |

| | | | | | |
|---|---|---|---|---|---|
| 8. | Na2SO4 | As needed | 142.04 | — | — |

To a solution of 2-Pentyn-1-ol (60 g, 0.71 mol) in THF (420 mL) cooled to −5° C., tosyl chloride (142.9 g, 0.75 mol) and KOH (79.9 g, 1.42 mol) were added and the reaction mixture was stirred at room temperature for 1 h. After completion of starting material, the reaction mixture was extracted with ethyl acetate (300 mL×2), washed with water (100 mL×2), brine (50 mL×2) and dried over Na₂SO₄. The combined organic extracts were evaporated under reduced pressure to obtain the crude product which was purified by column chromatography (100-200 mesh silica gel, 20% EtOAc-hexane) to furnish pent-2-ynyl 4-methylbenzenesulfonate (110 g, 64%) as a light red liquid.

### Preparation of Compound of Formula 3 (Octa-2,5-diyn-1-ol:)

The compound of Formula 2 obtained as described above is then coupled with propargyl alcohol in the presence of CuI, K₂CO₃ and TBAI to produce the compound represented by Formula 3. The yield of the compound ranges from 93-99%. The reaction scheme involved in this process is as follows:

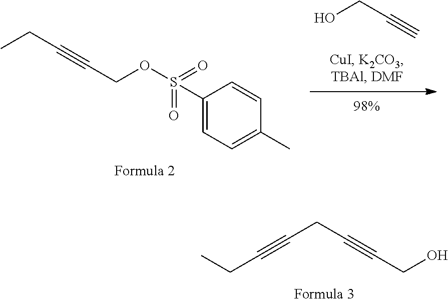

In an exemplary embodiment, the raw materials used for this step are illustrated in Table 2:

TABLE 2

| S. | Name of the | | | M. | | | |
|---|---|---|---|---|---|---|---|
| No. | Material | Qty. | | Wt. | Moles | Mole Ratio | |
| 1. | Compound | 60 | g | 84.5 | 0.71 | 1 | |
| | represented | | | | | | |
| | by Formula | | | | | | |
| | 2 | | | | | | |
| 2. | Propargyl | 15.52 | g | 56.06 | 0.27 | 0.38 | |
| | alcohol | | | | | | |
| 3. | Potassium | 47.8 | g | 138.2 | 0.34 | 0.48 | |
| | Carbonate | | | | | | |
| 4. | CuI | 43.9 | g | 190.45 | 0.23 | 0.32 | |
| 5. | TBAI | 85.30 | g | 369.37 | 0.23 | 0.32 | |
| 6. | DMF | 440 | mL | 73.09 | — | 7.33 | vol. |
| 7. | Ethyl | 2 × 300 | mL | 88.11 | — | 2 × 5 | vol. |
| | acetate | | | | | | |
| 8. | Cold water | 2 × 200 | mL | 18 | — | 2 × 3.33 | vol. |
| 9. | Brine | 2 × 100 | mL | — | — | 2 × 1.67 | vol. |

| | | | | | |
|---|---|---|---|---|---|
| 10. | Na₂SO₄, | As needed | 142.04 | — | — |
| | anhydrous | | | | |

To a stirred solution of potassium carbonate (47.8 g, 0.34 mol), CuI (43.9 g, 0.23 mol), and TBAI (85.30 g, 0.23 mol) in DMF (440 mL) cooled to 0° C., propargyl alcohol (15.52 g, 0.27 mol) was added portion wise at room temperature followed by compound represented by Formula 2 (55 g, 0.23 mol) and the reaction mixture was stirred at room temperature for 16 h. After completion of starting materials, the reaction mixture was cooled to 0° C. and diluted with cold water, ethyl acetate (300 mL×2), filtered through celite bed and washed with ethyl acetate. The combined organic extracts were washed with cold water (200 mL×2), brine (100 mL×2) and dried over anhydrous Na₂SO₄. Solvent was evaporated under reduced pressure to obtain the crude product which was purified by column chromatography (100-200 mesh silica gel, 20% EtOAc in hexane) to furnish octa-2,5-diyn-1-ol (55 g, 98%) as a light red liquid.

### Preparation of a Compound of Formula 4 (1-bromoocta-2,5-diyne)

The compound of Formula 3 obtained as described above is then brominated with PBr₃ to produce the compound represented by Formula 4. The reaction scheme involved in this process is as follows:

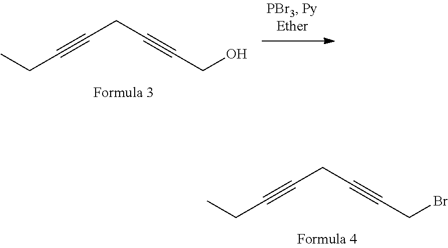

In an exemplary embodiment, the raw materials used for this step are illustrated in Table 3:

TABLE 3

| S. | Name of the | | | M. | | | |
|---|---|---|---|---|---|---|---|
| No. | Material | Qty. | | Wt. | Moles | Mole Ratio | |
| 1. | Compound | 55 | g | 122.22 | 0.45 | 1 | |
| | represented | | | | | | |
| | by Formula | | | | | | |
| | 3 | | | | | | |
| 2. | PBr₃ | 17.13 | mL | 270.69 | 0.18 | 0.4 | |
| 3. | Diethylether | 550 | mL | 74.12 | | 10 | vol. |
| 4. | Pyridine | 3.6 | mL | 79.1 | 0.04 | 0.009 | |
| 5. | Ethyl | 2 × 200 | mL | 88.11 | — | 2 × 3.63 | vol. |
| | acetate | | | | | | |
| 6. | Cold water | 100 | mL | 18 | — | 1.82 | vol. |
| 7. | Brine | 100 | mL | — | — | 1.82 | vol. |

| | | | | | |
|---|---|---|---|---|---|
| 8. | Na₂SO₄, | As needed | 142.04 | — | — |
| | anhydrous | | | | |

To a stirred solution of compound 3 (55 g, 0.45 mol) in diethylether (550 mL) cooled to 0° C., pyridine (3.6 mL, 0.04 mol), PBr₃ (17.13 mL, 0.18 mol) were added at 0° C. and the reaction mixture was stirred at room temperature for 16 h. After the completion of starting material, the reaction mixture was cooled to 0° C., diluted with cold water, and extracted with ethyl acetate (200 mL×2). The combined organic extracts were washed with cold water (100 mL×1), brine (100 mL×1), dried over anhydrous Na₂SO₄ and evaporated under reduced pressure to furnish 1-bromoocta-2,5-diyne (75 g, crude) as a red liquid which was carried to the next step without further purification.

### Preparation of a Compound of Formula 5 (undeca-2,5,8-triyn-1-ol)

The compound of Formula 4 obtained as described above is coupled with propargyl alcohol to produce the compound of Formula 5. The yield of the compound ranges from 52-62%. The reaction scheme involved in this process is as follows:

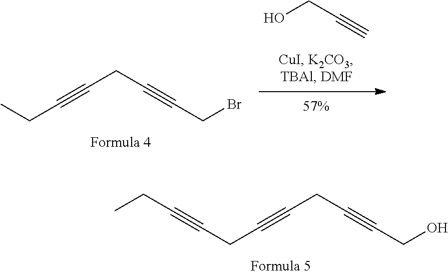

In an exemplary embodiment, the raw materials used for this step are illustrated in Table 4:

TABLE 4

| S. | Name of the | | | M. | | | |
|---|---|---|---|---|---|---|---|
| No. | Material | Qty. | | Wt. | Moles | Mole Ratio | |
| 1. | Compound | 75 | g | 187.5 | 0.40 | 1 | |
| | represented | | | | | | |
| | by Formula | | | | | | |
| | 4 | | | | | | |
| 2. | Propargyl | 27.2 | g | 56.06 | 0.48 | 1.2 | |
| | alcohol | | | | | | |
| 3. | Potassium | 83 | g | 138.2 | 0.60 | 1.5 | |
| | Carbonate | | | | | | |
| 4. | CuI | 77 | g | 190.45 | 0.40 | 1 | |
| 5. | TBAI | 149.5 | g | 369.37 | 0.40 | 1 | |
| 6. | DMF | 450 | mL | 73.09 | — | 6 | vol. |
| 7. | Ethyl | 300 | mL | 88.11 | — | 4 | vol. |
| | acetate | | | | | | |
| 8. | Cold water | 2 × 100 | mL | 18 | — | 2 × 1.33 | vol. |
| 9. | Brine | 100 | mL | — | — | 1.33 | vol. |

| | | | | | |
|---|---|---|---|---|---|
| 10. | Na₂SO₄ | As needed | 142.04 | — | — |

In an exemplary embodiment of this step, to a stirred solution of potassium carbonate (83 g, 0.60 mol), CuI (77 g, 0.40 mol) and TBAI (149.5 g, 0.40 mol) in DMF (450 mL) cooled to 0° C., propargyl alcohol (27.2 g, 0.48 mol) and compound represented by Formula 4 (75 g, 0.40 mol) were sequentially added and stirred at room temperature for 16 h. After the completion of starting materials, the reaction mixture was cooled to 0° C. and diluted with cold water, ethyl acetate (300 mL), filtered through a Celite™ pad using Buchner funnel and washed with ethyl acetate. The filtrate was taken and the organic layers were separated. The combined organic extracts were washed with cold water (100 mL×2), brine solution (100 mL×1), dried over Na₂SO₄ and evaporated under reduced pressure to obtain the crude product which was purified by column chromatography (100-200 mesh silica gel, 20% EtOAc in hexane) to furnish undeca-2,5,8-triyn-1-ol (37 g, 57%) as a pale yellow liquid.

### Preparation of a Compound of Formula 6 (1-bromoundeca-2,5,8-triyne)

The compound of Formula 5 obtained as described above is then brominated with PBr3 to produce the compound of Formula 6. The reaction scheme involved in this process is as follows:

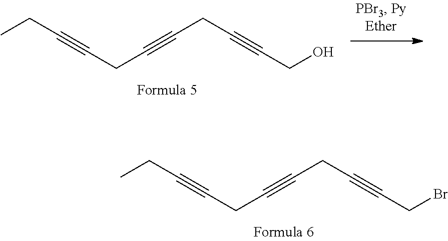

In an exemplary embodiment, the raw materials used for this step are illustrated in Table 5:

TABLE 5

| S. | Name of the | | | M. | | | |
|---|---|---|---|---|---|---|---|
| No. | Material | Qty. | | Wt. | Moles | Mole Ratio | |
| 1. | Compound | 37 | g | 160.87 | 0.23 | 1 | |
| | represented | | | | | | |
| | by Formula | | | | | | |
| | 5 | | | | | | |
| 2. | PBr₃ | 0.79 | mL | 270.69 | 0.09 | 0.39 | |
| 3. | Diethylether | 370 | mL | 74.12 | | | |
| 4. | Pyridine | 1.86 | mL | 79.1 | 0.02 | 0.09 | |
| 5. | Ethyl | 100 | mL | 88.11 | — | 2.7 | vol. |
| | acetate | | | | | | |
| 6. | Cold Water | 2 × 50 | mL | 18 | — | 2 × 1.35 | vol. |
| 7. | Brine | 50 | mL | — | — | 1.35 | vol. |

| | | | | | |
|---|---|---|---|---|---|
| 8. | Na₂SO₄ | As needed | 142.04 | — | — |

To a stirred solution of the compound represented by Formula 5 (37 g, 0.23 mol) in ether (370 mL) cooled to 0° C., pyridine (1.86 mL, 0.02 mol), PBr₃ (0.79 mL, 0.09 mol) were added at 0° C. and stirred at room temperature for 16 h. After the completion of starting material, the reaction mixture was cooled to 0° C. and diluted with cold water, and extracted with ethyl acetate (100 mL). The combined organic extracts were washed with cold water (50 mL×2), brine solution (50×1), dried over Na₂SO₄ and evaporated under reduced pressure to furnish 1-bromoundeca-2,5,8-triyne (42 g, crude) as a pale yellow color liquid which was carried to the next step without further purification.

### Preparation of Compound of Formula 7 (tetradeca-2,5,8,11-tetrayn-1-ol)

The compound of Formula 6 obtained as described above is coupled with propargyl alcohol to produce the compound of Formula 7. The yield of the compound ranges from 27-37%. The reaction scheme involved in this process is as follows:

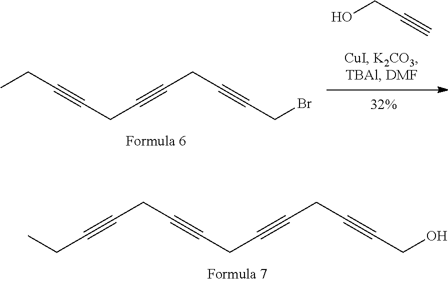

In an exemplary embodiment, the raw materials used for this step are illustrated in Table 6:

TABLE 6

| S. | Name of the | | | M. | | | |
|---|---|---|---|---|---|---|---|
| No. | Material | Qty. | | Wt. | Moles | Mole Ratio | |
| 1. | Compound | 42 | g | 233.33 | 0.18 | 1 | |
| | represented | | | | | | |
| | by Formula | | | | | | |
| | 4 | | | | | | |
| 2. | Propargyl | 14 | g | 56.06 | 0.25 | 1.39 | |
| | alcohol | | | | | | |
| 3. | Potassium | 38 | g | 138.2 | 0.27 | 1.5 | |
| | Carbonate | | | | | | |
| 4. | CuI | 35.85 | g | 190.45 | 0.18 | 1 | |
| 5. | TBAI | 69.5 | g | 369.37 | 0.18 | 1 | |
| 6. | DMF | 250 | mL | 73.09 | | 5.95 | vol. |
| 7. | Cold water | 200 | mL | 18 | | 4.76 | vol. |
| 8. | Ethyl | 200 | mL | 88.11 | | 4.76 | vol. |
| | acetate | | | | | | |
| 9. | Ethyl | 2 × 100 | mL | 88.11 | | 2 × 2.38 | vol. |
| | acetate | | | | | | |
| 10. | Cold Water | 2 × 50 | mL | 18 | | 2 × 1.19 | vol. |
| 12. | Brine | 50 | mL | — | | 1.19 | vol. |

| | | | |
|---|---|---|---|
| 11. | Na₂SO₄ | As needed | 142.04 |

In an exemplary embodiment of this step, to a solution of potassium carbonate (38 g, 0.27 mol), CuI (35.85 g, 0.18 mol) and TBAI (69.5 g, 0.18 mol) in DMF (250 mL) cooled to 0° C., propargyl alcohol (14 g, 0.25 mol) and the compound represented by Formula 6 (42 g, 0.18 mol) were added drop wise for 30 min and stirred for 16 h at room temperature. After the completion of starting material, the reaction mixture was cooled to 0° C. and diluted with cold water (200 mL), ethyl acetate (200 mL), filtered through Celite™ bed using Buchner funnel and washed with ethyl acetate (100 mL×2). The organic layers were separated and the combined organic extracts were washed with cold water (50 mL×2), brine solution (50 mL×1), dried over Na₂SO₄ and evaporated under reduced pressure to obtain the crude product which was purified by column chromatography (100-200 mesh silica gel, 20% EtOAc in hexane) to furnish tetradeca-2,5,8,11-tetrayn-1-ol (12 g, 32%) as a pale yellow solid.

### Preparation of a Compound of Formula 8 (1-bromotetradeca-2,5,8,11-tetrayne)

The Compound of Formula 7 obtained as described above is brominated with PBr₃ to produce the Compound of Formula 8. The yield of the compound ranges from 18-28%. The reaction scheme involved in this process is as follows:

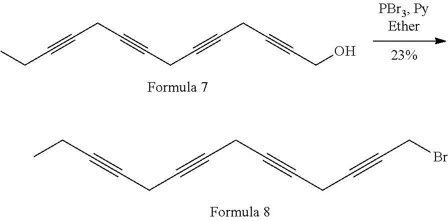

In an exemplary embodiment, the raw materials used for this step are illustrated in Table 7:

TABLE 7

| S. | Name of the | | | M. | | | |
|---|---|---|---|---|---|---|---|
| No. | Material | Qty. | | Wt. | mM | Mole Ratio | |
| 1. | Compound | 7.5 | g | 198.4 | 37.8 | 1 | |
| | represented | | | | | | |
| | by Formula 7 | | | | | | |
| 2. | PBr3 | 1.44 | mL | 270.69 | 15.15 | 0.4 | |
| 3. | Dichloro- | 75 | mL | 84.93 | | 10 | vol. |
| | methane | | | | | | |
| 4. | Pyridine | 0.3 | mL | 79.1 | 3.78 | 0.1 | |
| 5. | Dichloro- | 2 × 100 | mL | 84.93 | — | 2 × 13.33 | vol. |
| | methane | | | | | | |
| 6. | Water | 2 × 25 | mL | 18 | — | 2 × 3.33 | vol. |
| 7. | Brine | 2 × 25 | mL | — | — | 2 × 3.33 | vol. |

| | | | | | |
|---|---|---|---|---|---|
| 8. | Na2SO4 | As needed | 142.04 | — | — |

To a stirred solution of compound represented by Formula 7 (7.5 g, 37.8 mmol) in dry dichloromethane (75 mL), cooled to 0° C., pyridine (0.3 mL, 3.78 mmol) and PBr₃ (1.44 mL, 15.15 mmol) were added at 0° C., then the reaction mixture was stirred at room temperature for 16 h. After the completion of starting material, the reaction mixture was quenched with ice cold water and then extracted with dichloromethane (100 mL×2). The combined organic extracts were washed with water (25 mL×2), brine (25 mL×2), dried over Na₂SO₄ and evaporated under reduced pressure to obtain the crude product which was purified by column chromatography (100-200 mesh silica gel, 1% EtOAc in hexane) to furnish 1-bromotetradeca-2,5,8,11-tetrayne (2.3 g, 23%) as a yellow color solid.

### Preparation of a Compound of Formula 9 (heptadeca-2,5,8,11,14-pentayn-1-ol)

The compound of Formula 8 obtained as described above is coupled with ¹³C labeled propargyl alcohol to produce the compound of Formula 9. The yield of the compound ranges from 45-55%. The reaction scheme involved in this process is as follows:

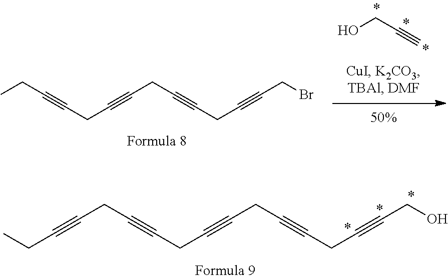

In an exemplary embodiment, the raw materials used for this step are illustrated in Table 8:

TABLE 8

| S. | Name of the | | | M. | | | |
|---|---|---|---|---|---|---|---|
| No. | Material | Qty. | | Wt. | mM | Mole Ratio | |
| 1. | Compound | 1.7 | g | 280.34 | 6.53 | 1 | |
| | represented | | | | | | |
| | by Formula 8 | | | | | | |
| 2. | ¹³C labeled | 0.36 | g | 56.06 | 6.42 | 0.98 | |
| | Propargyl | | | | | | |
| | alcohol | | | | | | |
| 3. | Potassium | 1.35 | g | 138.2 | 9.78 | 1.49 | |
| | Carbonate | | | | | | |
| 4. | CuI | 1.24 | g | 190.45 | 6.53 | 1 | |
| 5. | TBAI | 2.41 | g | 369.37 | 6.53 | 1 | |
| 6. | DMF | 14 | mL | 73.09 | — | 8.23 | vol. |
| 7. | Cold water | 10 | mL | 18 | — | 5.88 | vol. |
| 8. | Ethyl acetate | 2 × 50 | mL | 88.11 | — | 2 × 29.41 | vol. |
| 9. | Cold Water | 2 × 25 | mL | 18 | — | 2 × 14.7 | vol. |
| 10. | Brine | 25 | mL | — | — | 14.7 | vol. |

| | | | | | |
|---|---|---|---|---|---|
| 11. | Na₂SO₄ | As needed | 142.04 | — | — |

To a stirred solution of potassium carbonate (1.35 g, 9.78 mmol), CuI (1.24 g, 6.53 mmol) and TBAI (2.41 g, 6.53 mmol) in DMF (14 mL) cooled to 0° C., ¹³C labeled propargyl alcohol (0.36 g, 6.42 mmol) and the compound represented by Formula 8 (1.7 g, 6.53 mmol) were added drop wise and stirred at room temperature for 16 h. After completion of starting materials, the reaction mixture was cooled to 0° C. and diluted with cold water (10 mL), ethyl acetate (50 mL×2), filtered through a Celite™ pad using Buchner funnel and washed with ethyl acetate. The filtrate was taken and the organic layer was separated using a separating funnel. The combined organic extracts were washed with cold water (25 mL×2), brine solution (25 mL×1), dried over Na₂SO₄ and evaporated under reduced pressure to obtain the crude product which was purified by column chromatography (100-200 mesh silica gel, 16% EtOAc in hexane) to furnish heptadeca-2,5,8,11,14-pentayn-1-ol (750 mg, 50%) as a yellow solid.

### Preparation of Compound of Formula 10

The ¹³C labeled compound of Formula 9 obtained as described above is selectively reduced with Lindlar's Catalyst to produce the compound represented by Formula 10. The yield of the compound ranges from 63-73%. The reaction scheme involved in this process is as follows:

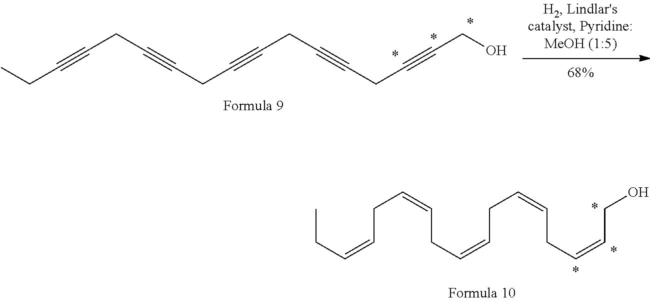 [SMILES: CCC#CCC#CCC#CCC#CCC#CCO ; CC/C=C\C/C=C\C/C=C\C/C=C\C/C=C\CO]

In an exemplary embodiment, the raw materials used for this step are illustrated in Table 9:

TABLE 9

| S. | Name of the | | | M. | | | |
|---|---|---|---|---|---|---|---|
| No. | Material | Qty. | | Wt. | mM | Mole Ratio | |
| 1. | Compound of | 1.4 | g | 239.31 | 5.85 | 1 | |
| | Formula 9 | | | | | | |
| 2. | Lindlar's | 1.44 | g | — | — | — | |
| | catalyst | | | | | | |
| 3. | Methanol/ | 24 | mL | — | — | 17.14 | vol. |
| | Pyridine (5:1) | | | | | | |
| 4. | Methanol | — | | 32 | — | — | |
| 5. | Ethyl acetate | 2 × 50 | mL | 88.11 | — | 2 × 35.71 | vol. |
| 6. | 1N HCl | 10 | mL | 36.5 | — | 7.14 | vol. |
| 7. | Brine | 10 | mL | — | — | 7.14 | vol. |

| | | | | | |
|---|---|---|---|---|---|
| 8. | Na₂SO₄ | As needed | 142.04 | — | — |

To a stirred solution of compound represented by Formula 9 (1.4 g, 5.85 mmol) in methanol/pyridine (5:1, 24 mL), Lindlar's catalyst (1.4 g, w/w) was added. The reaction mixture was stirred under H₂ atmosphere at room temperature for 16 h. After completion of starting material, the reaction mixture was filtered through a Celite™ pad and washed with methanol. The solvent was evaporated under reduced pressure and the crude obtained was extracted with ethyl acetate (50 mL×2), and washed with 1N HCl solution (10 mL×1), brine solution (10 mL×1) and dried over Na₂SO₄. The combined organic extracts were evaporated under reduced pressure to obtain the crude product which was purified by column chromatography (100-200 mesh silica gel, 10% EtOAc in hexane) to furnish compound represented by Formula 10 (1.0 g, 68%) as a colorless liquid.

### Preparation of a Compound of Formula 11

The compound of Formula 10 obtained as described above is brominated with PBr₃ to produce the compound of Formula 11. The reaction scheme involved in this process is as follows:

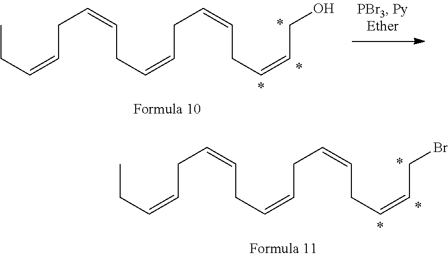 [SMILES: CC/C=C\C/C=C\C/C=C\C/C=C\C/C=C\CO ; CC/C=C\C/C=C\C/C=C\C/C=C\C/C=C\CBr]

In an exemplary embodiment, the raw materials used for this step are illustrated in Table 10:

TABLE 10

| S. | Name of the | | | M. | | | |
|---|---|---|---|---|---|---|---|
| No. | Material | Qty. | | Wt. | mM | Mole Ratio | |
| 1. | Compound of | 1.2 | g | 249.28 | 4.81 | 1 | |
| | Formula 10 | | | | | | |
| 2. | PBr3 | 0.52 | g | 270.69 | 1.92 | 0.4 | |
| 3. | Dichloromethane | 20 | mL | 84.93 | — | 16.67 | vol. |
| 4. | Pyridine | 0.38 | mL | 79.1 | 0.48 | 0.1 | |
| 5. | Cold water | 10 | mL | 18 | — | 8.33 | vol. |
| 6. | Dichloromethane | 2 × 50 | mL | 84.93 | — | 41.67 | vol. |
| 7. | Water | 15 | mL | 18 | — | 12.5 | vol. |
| 8. | Brine | 20 | mL | — | — | 16.67 | vol. |

| | | | | | |
|---|---|---|---|---|---|
| 9. | Na₂SO₄ | As needed | 142.04 | — | — |

To a solution of compound represented by Formula 10 (1.2 g, 4.81 mmol) in dry dichloromethane (20 mL) and pyridine (0.038 mL, 0.48 mmol) cooled to 0° C., PBr₃ (0.52 g, 1.92 mmol) was added drop wise and stirred at room temperature for 2 h. After completion of starting material, the reaction mixture was quenched with ice cold water (10 mL×1) and extracted with dichloromethane (50 mL×2). The combined organic extracts were washed with water (15 mL×1), brine (20 mL×1), dried over Na₂SO₄ and evaporated under reduced pressure to furnish compound represented by Formula 11 (1.2 g, crude) as a yellow liquid which was carried to the next step without further purification.

### Preparation of Compound of Formula 12

The compound of Formula 11 obtained as described above was coupled with methyl-pent-4-yonate to produce the compound represented by Formula 12. The yield of the compound ranges from 49-59%. The reaction scheme involved in this process is as follows:

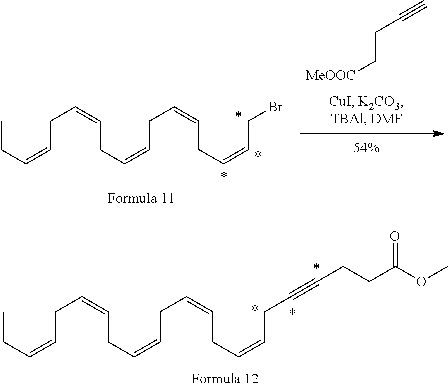

In an exemplary embodiment, the raw materials used for this step are illustrated in Table 11:

TABLE 11

| S. | Name of the | | | M. | | | |
|---|---|---|---|---|---|---|---|
| No. | Material | Qty. | | Wt. | mM | Mole Ratio | |
| 1. | Compound of | 200 | mg | 312.5 | 0.64 | 1 | |
| | Formula 11 | | | | | | |
| 2. | Methyl-pent- | 86 | mg | 111 | 0.76 | 1.19 | |
| | 4-yonate | | | | | | |
| 3. | Potassium | 132 | mg | 138.2 | 0.96 | 1.5 | |
| | Carbonate | | | | | | |
| 4. | CuI | 112 | mg | 190.45 | 0.64 | 1 | |
| 5. | TBAI | 236 | mg | 369.37 | 0.64 | 1 | |
| 6. | DMF | 10 | mL | 73.09 | — | 50 | vol. |
| 7. | Cold Water | 10 | mL | 18 | — | 50 | vol. |
| 8. | Diethyl ether | 2 × 25 | mL | 74.12 | — | 2 × 125 | vol. |
| 9. | Water | 10 | mL | 18 | — | 50 | vol. |
| 10. | Brine | 10 | mL | — | — | 50 | vol. |

| | | | | | |
|---|---|---|---|---|---|
| 11. | Na₂SO₄ | As needed | 142.04 | — | — |

To a solution of potassium carbonate (132 mg, 0.96 mmol), CuI (121 mg, 0.64 mmol) and TBAI (236 mg, 0.64 mmol) in dry DMF (10 mL) cooled to 0° C., methyl pent-4-ynoate (86 mg, 0.76 mmol) and the compound represented by Formula 11 (200 mg, 0.64 mmol) in DMF were added and stirred at room temperature for 16 h. After completion of starting material, the reaction mixture was quenched with ice cold water (10 mL) and filtered through a Celite™ bed and washed with diethyl ether (25 mL×2), water (10 mL×1), brine solution (10 mL×1) and dried over Na₂SO₄. The combined organic extracts were evaporated under reduced pressure to obtain the crude product which was purified by column chromatography (100-200 mesh silica gel, eluted at 2% EtOAc in hexane) to furnish compound represented by Formula 12 (120 mg, 54%) as a colorless liquid.

### Preparation of a Compound of Formula 13

The compound of Formula 12 obtained as described above was selectively reduced with Lindlar's catalyst to produce the compound of Formula 13. The yield of the compound ranges from 75-85%. The reaction scheme involved in this process is as follows:

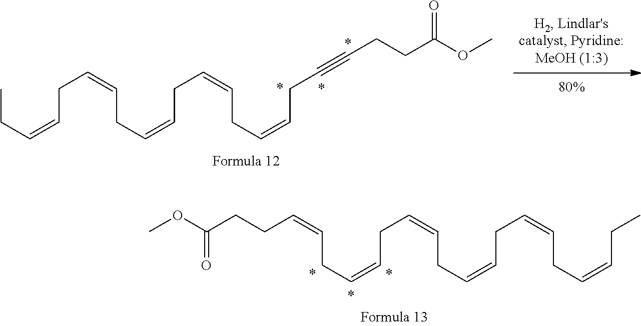

In an exemplary embodiment, the raw materials used for this step are illustrated in Table 12:

TABLE 12

| S. | Name of the | | | M. | | | |
|---|---|---|---|---|---|---|---|
| No. | Material | Qty. | | Wt. | mM | Mole Ratio | |
| 1. | Compound of | 500 | mg | 344.82 | 1.45 | 1 | |
| | Formula 12 | | | | | | |
| 2. | Lindlar's | 500 | mg | — | — | — | |
| | catalyst | | | | | | |
| 3. | Methanol/ | 10 | mL | — | — | 20 | vol. |
| | Pyridine (4:1) | | | | | | |
| 4. | Methanol | 20 | mL | 32 | — | 40 | vol. |
| 5. | Ethyl acetate | 2 × 30 | mL | 88.11 | — | 2 × 60 | vol. |
| 6. | 1N HCl | 10 | mL | 36.5 | — | 20 | vol. |
| 7. | Brine | 15 | mL | — | — | 30 | vol. |

| | | | | | |
|---|---|---|---|---|---|
| 8. | Na₂SO₄ | As needed | 142.04 | — | — |

To a solution of compound represented by Formula 12 (500 mg, 1.45 mmol) in dry methanol/pyridine (10 mL, 4:1), Lindlar's catalyst (500 mg, w/w) was added. The reaction mixture was stir under H₂ atmosphere at room temperature for 16 h. Additionally, Lindlar's catalyst (250 mg) was added two times at 4 h interval and reaction mixture was stirred under H₂ atmosphere. The reaction mixture was filtered through a Celite™ pad, washed with methanol (20 mL) and evaporated under reduced pressure. The crude obtained was extracted with ethyl acetate (30 mL×2), washed with 1N HCl solution (10 mL×1), brine solution (15 mL×1) and dried over Na₂SO₄. The combined organic layer was evaporated under reduced pressure to furnish compound represented by Formula 13 (400 mg, 80%) as a pale yellow liquid.

### Preparation of ¹³C Labeled DHA as Represented by Formula A

In the last step of the 13-step synthetic process, ¹³C labeled DHA of Formula A is obtained by ester hydrolysis of the compound represented by Formula 13 in the presence of lithium hydroxide. The yield of the compound ranges from 82-92%. The reaction scheme involved in this process is as follows:

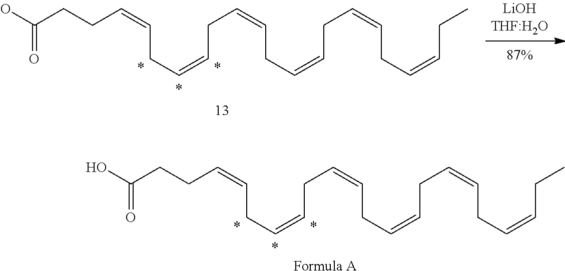

In an exemplary embodiment, the raw materials used for this step are illustrated in Table 13:

TABLE 13

| S. | Name of the | | | M. | | | |
|---|---|---|---|---|---|---|---|
| No. | Material | Qty. | | Wt. | mM | Mole Ratio | |
| 1. | Compound | 180 | mg | 346.15 | 0.52 | 1 | |
| | of Formula | | | | | | |
| | 13 | | | | | | |
| 2. | Lithium | 109 | mg | 23.95 | 2.6 | 5 | |
| | Hydroxide | | | | | | |
| 2. | THF/H₂O | 6 | mL | — | — | 33.33 | vol. |
| | (3:1) | | | | | | |
| 3. | Ethyl | 2 × 30 | mL | 88.11 | — | 2 × 166.67 | vol. |
| | acetate | | | | | | |
| 4. | Water | 10 | mL | 18 | — | 55.55 | vol. |
| 5. | Brine | 10 | mL | — | — | 55.55 | vol. |

| | | | | | |
|---|---|---|---|---|---|
| 6. | Na₂SO₄ | As needed | 142.04 | — | — |

To a solution of compound represented by Formula 13 (180 mg, 0.52 mmol) in THF/H₂O (6 mL, 3:1 ratio), lithium hydroxide (109 mg, 2.6 mmol) was added and stirred at room temperature for 16 h. After completion of starting material, the reaction mixture was quenched with aqueous citric acid solution; pH was adjusted to 4 and extracted with ethyl acetate (30 ml×2). The combined organic extracts were washed with water (10 mL×1), brine solution (10 mL×1) and dried over Na₂SO₄. The combined organic extracts were evaporated under reduced pressure to obtain the crude product which was purified by column chromatography (100-200 mesh silica gel, the product eluted at 15% EtOAc in hexane) to furnish the compound represented by Formula A (¹³C DHA) (150 mg, 87%) as a pale yellow liquid.

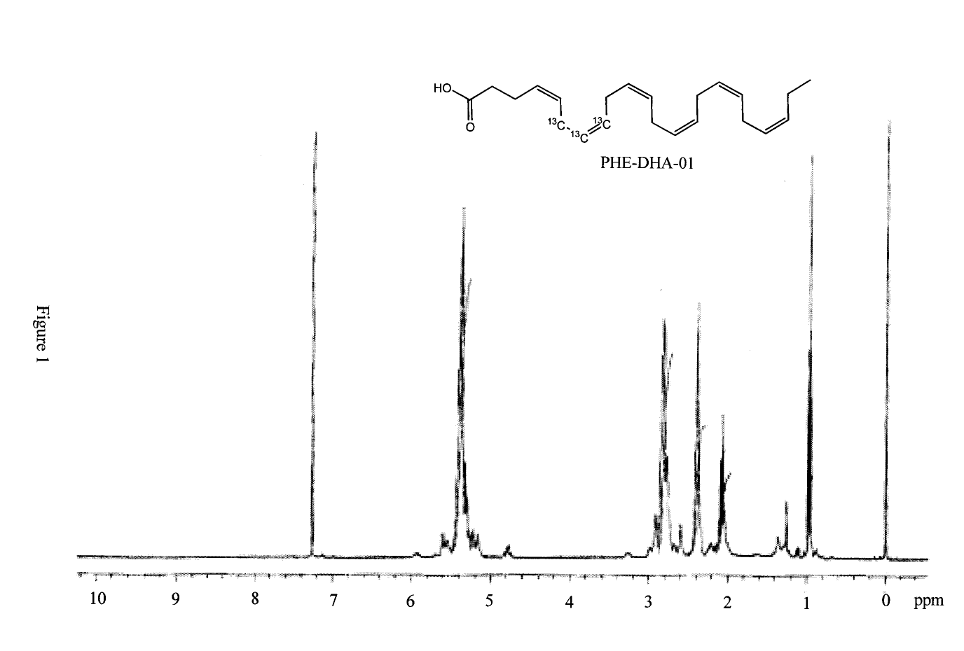

The identity of the Compound of Formula A produced by the synthetic process described above was ascertained by NMR spectroscopy. The NMR spectra obtained is presented in FIG. 1.

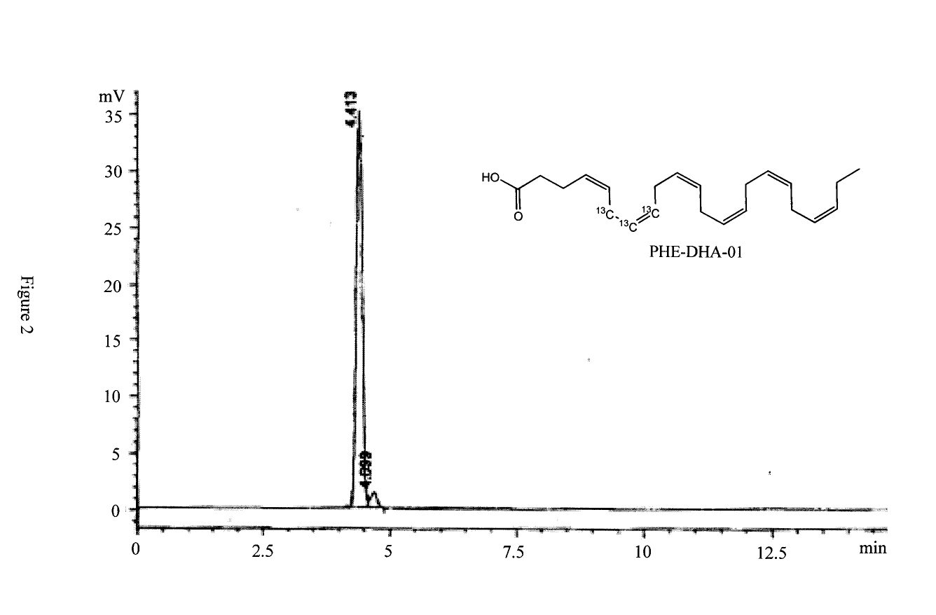

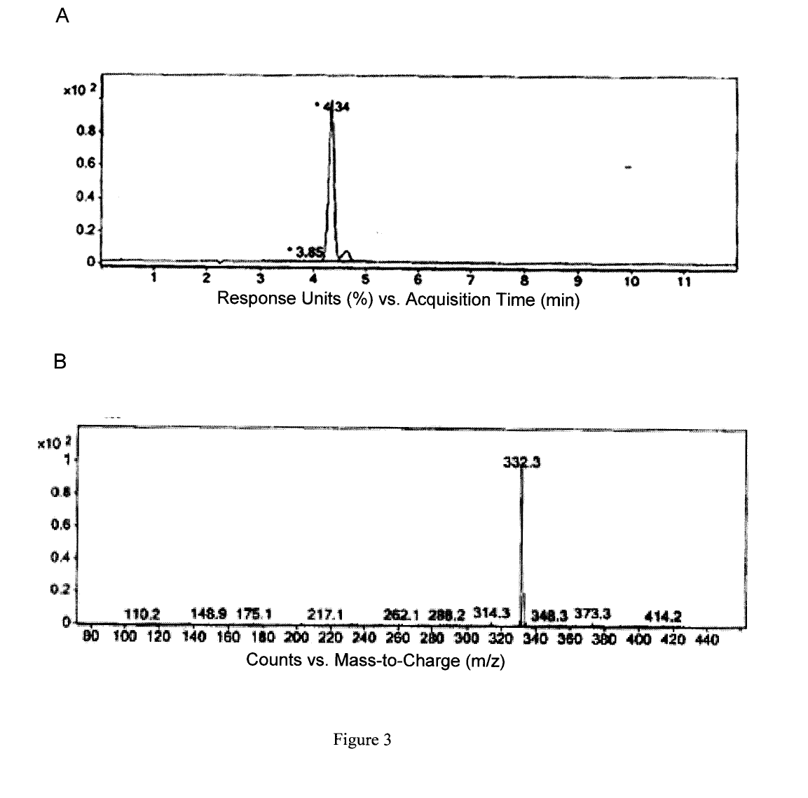

Purity of the sample obtained was determined by LC (See FIG. 2) and identity was further characterized by LC-MS (See FIGS. 3A and 3B). The purity of the sample was found to be 90%.

### Example 2

### Synthesis of ¹³C DHA by 12-Step Chemical Synthetic Process

An exemplary embodiment of the 12-step chemical synthesis process for preparing ¹³C DHA is shown in Scheme D:

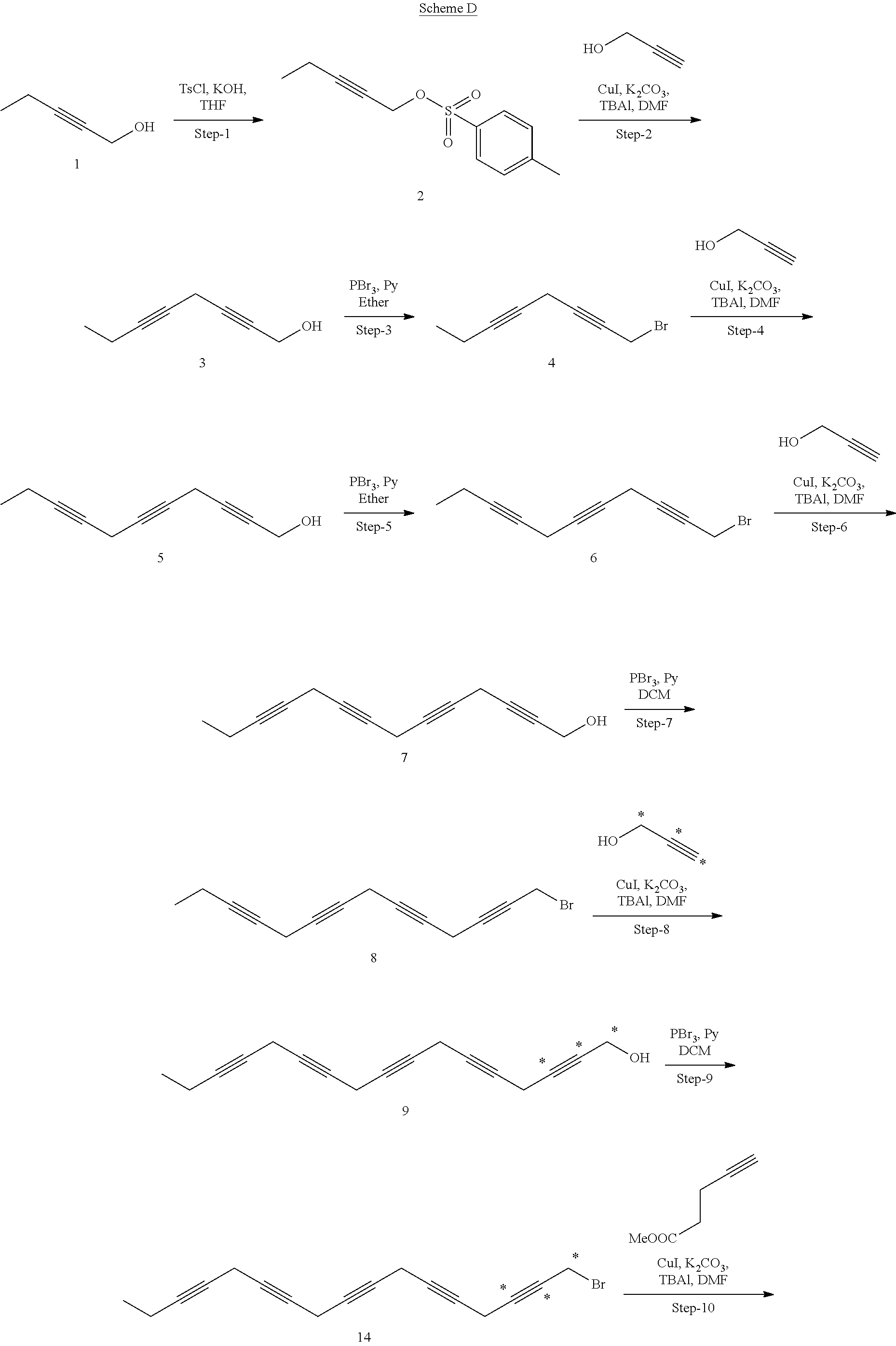

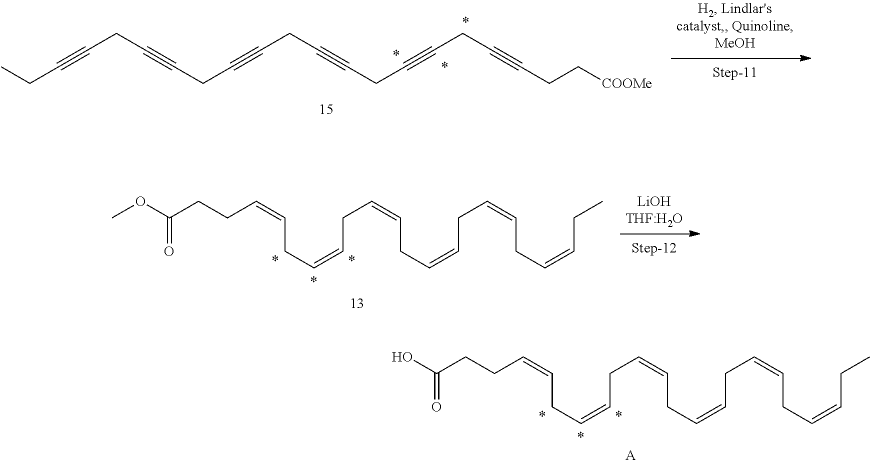

In this alternate 12-step synthesis strategy, the steps leading to the formation of the compound represented by Formula 9 are similar to those described in Example 1. The compound of Formula 9 thus produced is reacted with PBr₃ in the presence of Py and DCM to produce the compound represented by Formula 14. The compound of Formula 14 is coupled with methyl-pent-4-yonate in the presence of CuI, K₂CO₃ and TBAI in DMF to produce the compound of Formula 15. The compound of Formula 15 is then selectively hydrogenated in a H₂ atmosphere using a catalyst, e.g. Lindlar's catalyst, in the presence of quinoline and MeOH. The reaction is carried out at about room temperature. The selective reduction of the compound represented by Formula 15 results in the production of the compound represented by Formula 13. In the last step of the alternate 12-step synthetic process, ¹³C DHA of Formula A is obtained by ester hydrolysis of the compound represented by Formula 13 in the presence of lithium hydroxide, and in the presence of THF/H₂O.

It will be apparent to a person having skill in the art that all the common steps of this alternate 12-step strategy can be carried out under similar conditions as those described in Example 1 above.

The preferred embodiments of the invention described above are merely exemplary of the invention, which can be embodied in various forms. Therefore, specific details relating to the reagents and reaction conditions disclosed herein are not to be interpreted as limiting, but merely as an example.

It will also be apparent to a person skilled in the art that a number of variations and modifications can be made without departing from the scope of the invention as defined in the claims.

1. A process for preparing a ¹³C labeled fatty acid represented by Formula (i): 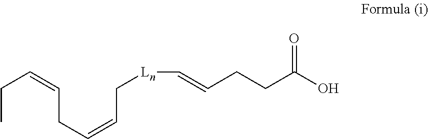
wherein L is —[CH═CH—CH₂]—, n is 0 to 6, and the fatty acid comprises at least one ¹³C labeled carbon residue, the process comprising:
(a) converting 2-pentyn-1-ol into a tosylate of Formula (ii): 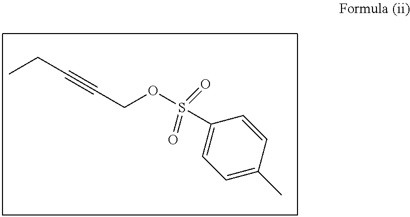 [SMILES: CCC#CCOS(=O)(=O)c1ccc(C)cc1]
(b) reacting the compound of Formula (ii) with propargyl alcohol in a coupling reaction, and optionally carrying out one or more additional steps of brominating followed by coupling with propargyl alcohol, to obtain a compound represented by Formula (iii): 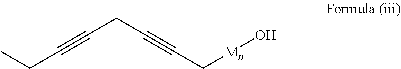
wherein M is —[C≡C—CH₂]—, and n is as defined above,
(c) carrying out a selective reduction of the compound represented by Formula (iii) to obtain a compound represented by Formula (iv): 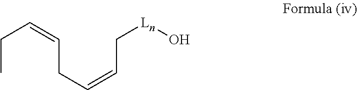
wherein L and n are as defined above,
(d) brominating the compound of Formula (iv) to produce a compound represented by Formula (v): 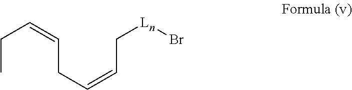
wherein L and n are as defined above,
(e) coupling the compound represented by Formula (v) with methyl pent-4-ynoate to obtain a compound represented by Formula (vi): 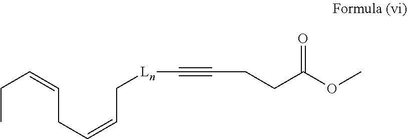
(f) carrying out a selective reduction of the compound represented by Formula (vi) to obtain a compound represented by Formula (vii): 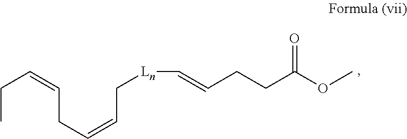
and
(g) ester-hydrolyzing the compound represented by Formula (vii) to obtain the compound represented by Formula (i),
wherein the propargyl alcohol used in at least one of the coupling reactions carried out in (b) is labeled with ¹³C at C₁, C₂, or C₃ of the propargyl alcohol, or a combination thereof.

2. The process of claim 1, wherein n is 1 to 4.

3. The process of claim 1, wherein n is 3.

4. The process of claim 1, wherein step (a) comprises reacting the 2-pentyn-1-ol with tosyl chloride (TsCl) to obtain the tosylate of Formula (ii).

5. The process of claim 1, wherein step (b) comprises carrying out three additional steps of brominating followed by coupling with propargyl alcohol, to obtain a compound represented by Formula (9): 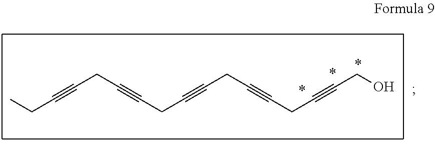 [SMILES: CCC#CCC#CCC#CCC#CCC#CCO]
wherein the compound is ¹³C labeled at one or more carbon atoms marked with an asterisk.

6. The process of claim 1, wherein the brominating reactions carried out in steps (b) and (d) comprise reacting the compound with PBr₃.

7. The process of claim 1, wherein the propargyl alcohol used in at least one of the coupling reactions carried out in (b) is labeled with ¹³C at C₁, C₂, and C₃ of the propargyl alcohol.

8. The process of claim 1, wherein the propargyl alcohol used in one of the coupling reactions carried out in (b) is labeled with ¹³C at C₁, C₂, and C₃ of the propargyl alcohol.

9. The process of claim 1, wherein step (b) comprises carrying out three additional steps of brominating followed by coupling with propargyl alcohol, and the propargyl alcohol used in the final coupling reaction is labeled with ¹³C at C₁, C₂, and C₃ of the propargyl alcohol, to obtain a compound represented by Formula (9): 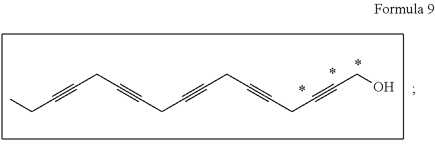 [SMILES: CCC#CCC#CCC#CCC#CCC#CCO]
wherein the compound is ¹³C labeled at all three carbon atoms marked with an asterisk.

10. The process of claim 1, wherein n is 3, and the fatty acid obtained is represented by Formula A:

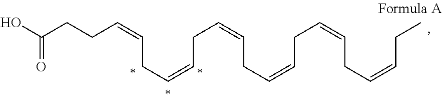 [SMILES: CC/C=C\C/C=C\C/C=C\C/C=C\C/C=C\C/C=C\CCC(=O)O]

11. A process for preparing a compound of Formula A 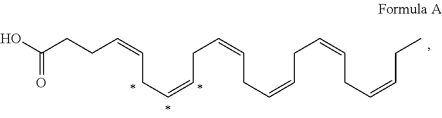 [SMILES: CC/C=C\C/C=C\C/C=C\C/C=C\C/C=C\C/C=C\CCC(=O)O]
wherein the compound is ¹³C labeled at one or more carbon atoms marked with an asterisk, the process comprising the steps of:
(a) protecting the primary alcohol of a 2-pentyn-1-ol of Formula 1: 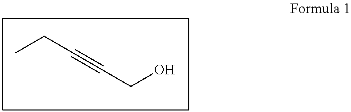
using a protecting agent to obtain a compound represented by Formula 2: 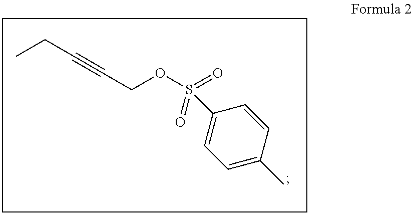 [SMILES: CCC#CCOS(=O)(=O)c1ccc(C)cc1]
(b) coupling the compound represented by Formula 2 with propargyl alcohol to obtain a compound represented by Formula 3: 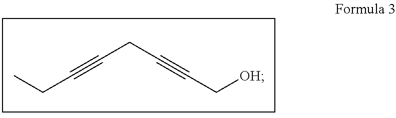
(c) brominating the compound represented by Formula 3 to obtain a compound represented by Formula 4: 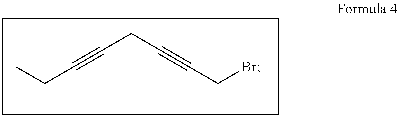
(d) coupling the compound represented by Formula 4 with propargyl alcohol to obtain a compound represented by Formula 5 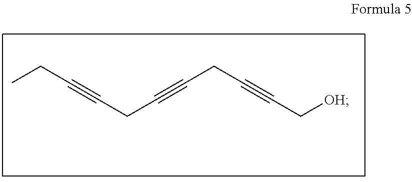
(e) brominating the compound represented by Formula 5 to obtain a compound represented by Formula 6: 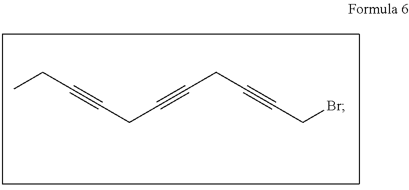
(f) coupling the compound represented by Formula 6 with propargyl alcohol to obtain a compound represented by Formula 7: 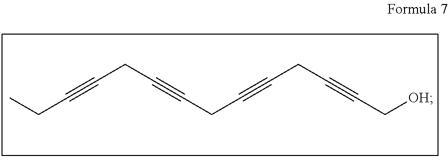
(g) brominating the compound represented by Formula 7 to obtain a compound represented by Formula 8: 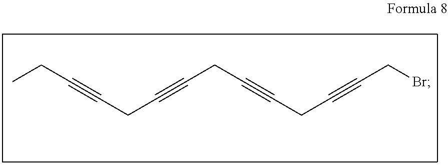
(h) coupling the compound represented by Formula 8 with ¹³C labeled propargyl alcohol to yield a compound represented by Formula 9: 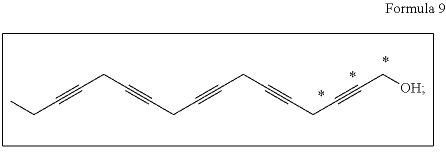 [SMILES: CCC#CCC#CCC#CCC#CCC#CCO]
wherein the compound is ¹³C labeled at one or more carbon atoms marked with an asterisk,
(i) selectively reducing the compound represented by Formula 9 to obtain a compound represented by Formula 10: 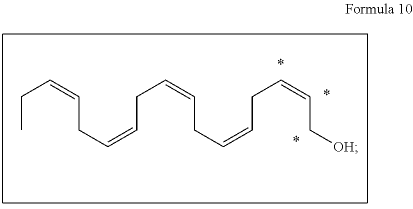 [SMILES: CC/C=C\C/C=C\C/C=C\C/C=C\C/C=C\CO]
(j) brominating the compound represented by Formula 10 to obtain a compound represented by Formula 11: 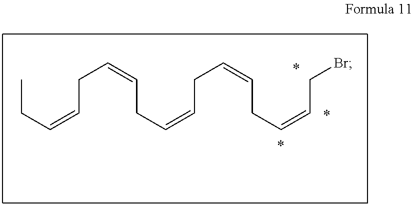 [SMILES: CC/C=C\C/C=C\C/C=C\C/C=C\C/C=C\CBr]
(k) coupling the compound represented by Formula 11 with methyl pent-4-ynoate to obtain a compound represented by Formula 12: 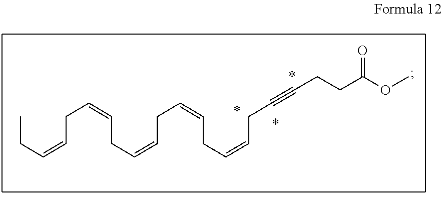 [SMILES: CC/C=C\C/C=C\C/C=C\C/C=C\C/C=C\CC#CCCC(=O)OC]
(l) selectively reducing the compound represented by Formula 12 to obtain a compound represented by Formula 13: 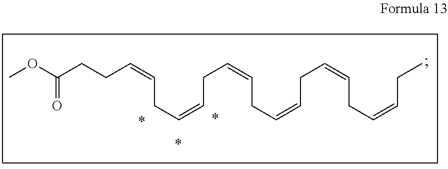 [SMILES: CC/C=C\C/C=C\C/C=C\C/C=C\C/C=C\C/C=C\CCC(=O)OC]
and
(m) ester-hydrolyzing the compound represented by Formula 13 to yield the compound represented by Formula A.

12. The process as claimed in claim 11, wherein the step (a) of protecting the primary alcohol of the 2-pentyn-1-ol comprises reacting the 2-pentyn-1-ol with tosyl chloride (TsCl) and KOH.

13. The process as claimed in claim 12, wherein the step (a) is carried out at a temperature of between about −5° C. to about room temperature.

14. The process as claimed in claim 11, wherein the coupling reaction of step (b) is conducted in presence of K₂CO₃, CuI, tetrabutylammonium iodide (TBAI) and N,N-dimethylformamide (DMF).

15. The process as claimed in claim 14, wherein the coupling reaction of step (b) is carried out at a temperature of between about 0° C. to about room temperature.

16. The process as claimed in claim 11, wherein the brominating step (c) comprises reacting the compound represented by Formula 3 with PBr₃ in the presence of diethyl ether and pyridine.

17. The process as claimed in claim 16, wherein the brominating step (c) is carried out at a temperature of between about 0° C. to about room temperature.

18. The process as claimed in claim 11, wherein the coupling reaction of step (d) is carried out in the presence of K₂CO₃, CuI, tetrabutylammonium iodide (TBAI) and N,N-dimethylformamide (DMF).

19. The process as claimed in claim 18, wherein the coupling reaction of step (d) is carried out at a temperature of between about 0° C. to about room temperature.

20. The process as claimed in claim 11, wherein the brominating step (e) comprises reacting the compound represented by Formula 5 with PBr₃ in the presence of diethylether and pyridine.

21. The process as claimed in claim 20, wherein the brominating step (e) is carried out at a temperature of between about 0° C. to about room temperature.

22. The process as claimed in claim 11, wherein the coupling reaction of step (f) is carried out in the presence of K₂CO₃, CuI, tetrabutylammonium iodide (TBAI) and N,N-dimethylformamide (DMF).

23. The process as claimed in claim 22, wherein the coupling reaction of the step (f) is carried out at a temperature of between about 0° C. to about room temperature.

24. The process as claimed in claim 11, wherein the brominating step (g) comprises reacting the compound represented by Formula 7 with PBr₃ in the presence of diethylether and pyridine.

25. The process as claimed in claim 24, wherein the brominating step (g) is carried out at a temperature of between about 0° C. to about room temperature.

26. The process as claimed in claim 11, wherein the coupling reaction of step (h) is carried out in the presence of K₂CO₃, CuI, tetrabutylammonium iodide (TBAI) and N,N-dimethylformamide (DMF).

27. The process as claimed in claim 26, wherein the coupling reaction of step (h) is carried out at a temperature of between about 0° C. to about room temperature.

28. The process as claimed in claim 11, wherein the selective reduction of step (i) is carried out in a H₂ atmosphere at about room temperature using Lindlar's catalyst.

29. The process as claimed in claim 11, wherein the brominating step (j) comprises reacting the compound represented by Formula 10 with PBr₃ in the presence of diethylether and pyridine.

30. The process as claimed in claim 29, wherein the brominating step (j) is carried out at a temperature of between about 0° C. to about room temperature.

31. The process as claimed in claim 11, wherein the coupling reaction of step (k) is carried out in the presence of K₂CO₃, CuI, tetrabutylammonium iodide (TBAI) and N,N-dimethylformamide (DMF).

32. The process as claimed in claim 31, wherein coupling reaction of step (k) is carried out at a temperature of between about 0° C. to about room temperature.

33. The process as claimed in claim 11, wherein the selective reduction of step (1) is carried out in a H₂ atmosphere at about room temperature using Lindlar's catalyst.

34. The process as claimed in claim 11, wherein the step (m) of ester hydrolyzing the compound of Formula 13 is carried out in presence of LiOH.

35. The process as claimed in claim 34, wherein the step (m) is carried out at about room temperature.

36. A compound of Formula (i): 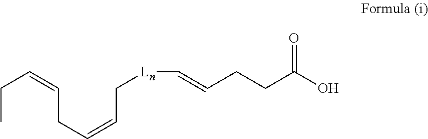
wherein L is —[CH═CH—CH₂]—, n is 0 to 6, and the fatty acid comprises at least one ¹³C labeled carbon residue.

37. The compound of claim 36, wherein n is 3.

38. The compound of claim 36, wherein the compound is as represented by Formula A: 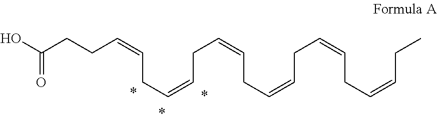 [SMILES: CC/C=C\C/C=C\C/C=C\C/C=C\C/C=C\C/C=C\CCC(=O)O]
wherein the compound is ¹³C labeled at one or more carbon atoms marked with an asterisk.

39. The compound of Formula (i), prepared by the process as claimed in claim 1.

40.-41. (canceled)

42. A reference marker for use in metabolic studies comprising a compound of Formula (i): 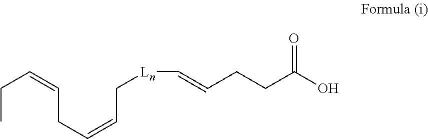
wherein L is —[CH═CH—CH₂]—, n is 0 to 6, and the compound comprises at least one ¹³C labeled carbon residue.

43. The reference marker of claim 42, wherein the compound is as represented by Formula A: 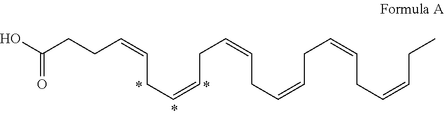 [SMILES: CC/C=C\C/C=C\C/C=C\C/C=C\C/C=C\C/C=C\CCC(=O)O]
wherein the compound is ¹³C labeled at one or more carbon atoms marked with an asterisk.

44. A process for preparing a compound of Formula A 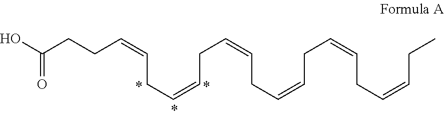 [SMILES: CC/C=C\C/C=C\C/C=C\C/C=C\C/C=C\C/C=C\CCC(=O)O]
wherein the compound is ¹³C labeled at one or more carbon atoms marked with an asterisk, the process comprising the steps of:
protecting the primary alcohol of a 2-pentyn-1-ol of Formula 1: 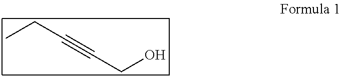
using a protecting agent to obtain a compound represented by Formula 2: 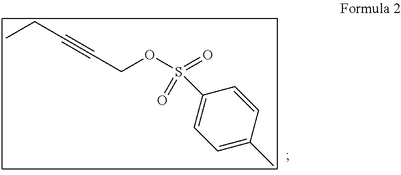 [SMILES: CCC#CCOS(=O)(=O)c1ccc(C)cc1]
coupling the compound represented by Formula 2 with propargyl alcohol to obtain a compound represented by Formula 3: 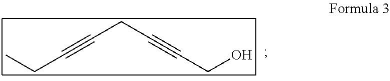
brominating the compound represented by Formula 3 to obtain a compound represented by Formula 4: 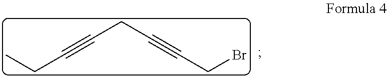
coupling the compound represented by Formula 4 with propargyl alcohol to yield a compound represented by Formula 5: 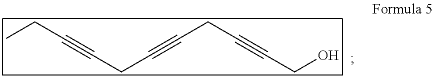
brominating the compound represented by Formula 5 to obtain a compound represented by Formula 6: 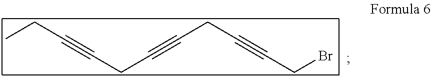
coupling the compound represented by Formula 6 with propargyl alcohol to obtain a compound represented by Formula 7: 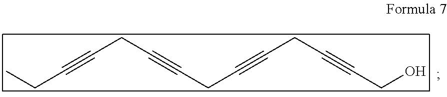
brominating the compound represented by Formula 7 to a compound represented by Formula 8: 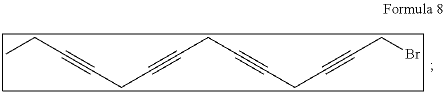
coupling the compound represented by Formula 8 with ¹³C labeled propargyl alcohol to yield a compound represented by Formula 9: 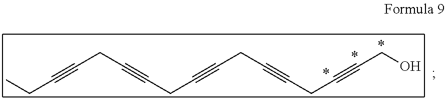 [SMILES: CCC#CCC#CCC#CCC#CCC#CCO]
wherein the compound is ¹³C labeled at one or more carbon atoms marked with an asterisk,
brominating the compound represented by Formula 9 to obtain a compound represented by Formula 10: 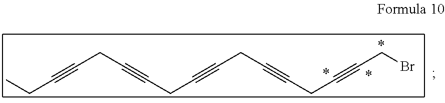 [SMILES: CCC#CCC#CCC#CCC#CCC#CCBr]
coupling the compound represented by Formula 10 with methyl pent-4-ynoate to yield a compound represented by Formula 11: 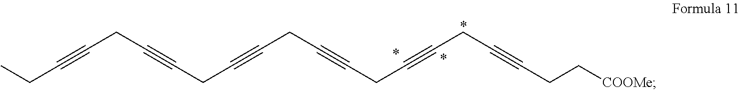 [SMILES: CCC#CCC#CCC#CCC#CCC#CCC#CCCC(=O)OC]
selectively reducing the compound represented by Formula 11 to yield a compound represented by Formula 12: 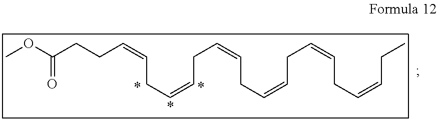 [SMILES: CC/C=C\C/C=C\C/C=C\C/C=C\C/C=C\C/C=C\CCC(=O)OC]
and
ester-hydrolyzing the compound represented by Formula 12 to yield the compound represented by Formula A.

45. A compound of Formula A prepared by the process as claimed in claim 44.
